(19) United States
(12) Patent Application Publication
(10) Pub. No.: US 2004/0014215 A1
(43) Pub. Date: Jan. 22, 2004

(54) Synchronised arabidopsis cell suspensions and uses thereof

(76) Inventors: Margit Menges, Fritzlar (DE); James Murray, Cambridge (GB)

Correspondence Address: Craig J Arnold Elie H Gendloff Alan D Miller, Amster Rothstein & Ebenstein, 90 Park Avenue, New York, NY, 10016, US

(21) Appl. No.: 10/333,005

(22) PCT Filed: Jul. 20, 2001
(86) PCT No.: PCT/EP01/08414

(30) Foreign Application Priority Data
Jul. 27, 2000 (GB) 0018305.3

(51) Int. Cl. C12N 5/04

(52) U.S. Cl. 435/419

(57) ABSTRACT

Improved methods are provided to obtain synchronised Arabidopsis cell suspension cultures. Also provided are synchronised Arabidopsis cell suspension cultures which may be obtained by the described methods, as well as uses of such suspension cultures to isolate and analyse cell cycle regulated or proteins.

This invention relates to the field of plant biotechnology. In particular this invention relates to methods for obtaining synchronised Arabidopsis cell suspension cultures as well as the use of such suspension cultures to isolate and analyse cell cycle regulated genes or proteins.

### BACKGROUND

Dispersed plant cell suspension cultures offer the possibility to study cell division in the absence of developmental processes and represent a potential useful system to investigate the cell cycle for basic and applied research. Their particular value is that they provide a homogeneous population of near identical cells that offer material for synchronisation by various procedures.

In an ideal synchronous culture, each cell should progress through the cell cycle at the same time as all the others and the behaviour of the whole population reflects the behaviour of any individual cell within the suspension culture. Taking samples at various time points in a synchronised culture for mRNA or protein analysis should provide material representative of specific cell cycle processes.

The cell cycle consists of four phases which normally follow each other in the order: G1-phase (Gap-1), S-phase (when DNA replication occurs), G2-phase (Gap-2) and mitosis (time of chromosome condensation, chromosome segregation and cell division).

Synchrony of cells in a culture is often measured by counting the proportion of the total number of cells that are engaged in mitosis at a particular time, as observed by microscopic examination of cells stained with a fluorescent DNA binding dye. When expressed as a percentage, this number is defined as the [symbol] mitotic index [symbol] . The maximum value reached is defined as the [symbol] peak mitotic index [symbol] .

Alternatively, the proportion of total cells engaged in S phase can be determined by methods that identify cells undergoing DNA replication. When expressed as a percentage, this is defined as the “labelling index”. The maximum value reached is defined as the peak labelling index.

Yet another alternative to identify the level of synchrony is to determine the proportion of cells at different phases of the cell cycle by flow cytometry (Galbraith et al., 1991, Juan et al. 1998.) which allows an estimate of the proportion of cells in G1-phase, S-phase and G2/M-phase.

In general, there are rather few plant cell cultures that can be synchronised to a high degree. Exceptions to this general rule, include the tobacco cell culture derived from the cultivar Bright Yellow-2 (BY-2 suspension culture; Nagata et al. 1992), a culture of Cantharantus roseus cells (Ito et al. 1997) and a suspension culture of alfalfa (Magyar et al., 1997).

Varying degrees of synchrony in plant cell suspension cultures have been achieved by starving the cells for phosphate, hormones or sucrose and subsequent readdition, as well by reversible blocking different stages of the cell cycle by various chemicals (Binarova et al. 1998, Fukuda et al. 1994, Riou-Khamlichi et al. 1999).

The tobacco BY-2 cell line, can be synchronised to a high degree (Nagata et al. 1992, Samuals et al. 1998). Using the drug aphidicolin (a DNA polymerase alpha inhibitor), which arrests cells in late G1/early S-phase, a mitotic index of 45-50% can be achieved in BY-2.

Much higher levels of mitotic synchrony could be achieved by treating the pre-synchronised BY-2 cells (after aphidicolin treatment) with propyzamide (an antitubulin drug) that induces mitotic arrest (Nagata et al. 1992, Reichfeld et al. 1999). Providing the propyzamide treatment is applied for a few hours only, the cell cycle restarts after propyzamide is removed and a mitotic index of up to 80-90% can be achieved. These so-called double block methods can therefore be used to increase the degree of synchronicity obtained.

Other drugs which can reversible arrest cells either in G1 or G2 are the chemical roscovitine or related compounds, which have been demonstrated to inhibit the histone H1 kinase activity of purified CDK/cyclin complexes (Meijer 1996, Planchais et al. 1997). Roscovitine is a member of the C2,N6,N9-substituted adenine family, like olomoucine, and these compounds and other various purine analogues, like bohemine have been shown to act as competitive inhibitors for ATP binding in enzymological studies (Planchais et al. 1997, Binarova et al. 1998). Mimosine, a rare plant amino acid, which is capable of blocking cell cycle progression has been demonstrated to be an effective reversible inhibitor of DNA replication in mammalian cells (Kalejta et al. 1997, Kulp et al. 1996). Use of this reagent in plant cells derived from a petunia protoplast culture, resulted in reversible block during G1 and in early/mid S-phase (Perennes et al. 1993).

Double phosphate starvation or auxin starvation of Catharanthus roseus cells (Madagascar periwinkle) produced synchronous cell division, where 70-80% of cells divide during a 3 to 4 hr period (Ito et al. 1997). This cell culture was used to isolate cDNAs for two mitotic cyclins, named CYS (A-type like cyclin) and CYM (B-type like cyclin).

Magyar et al. (1997) analysed the expression of four CDC2 homologues in alfalfa by using a synchronised cell line, which was blocked with aphidicolin for 36 h and released. Following this procedure a synchrony with a peak mitotic index of up to 40% could be achieved in this alfalfa-cell suspension.

Few Arabidopsis cell suspension cultures are available, and it seems to be difficult to achieve synchrony in Arabidopsis cell suspension cultures. Partial synchrony in an Arabidopsis cell suspension culture has been achieved by phosphate starvation in Gamborg B5 medium for 32 hours followed by aphidicolin block and release by washing and resuspending into fresh medium containing 20% (v/v) conditioned culture medium (Callard et al. 1997). Following this procedure, 2 h after release of block 50 to 60% of cells were found to be in S-phase and 50% were found to be in G2 phase after 8 h. However synchrony was reported to be lost before mitosis and synchronous re-entry into a second S-phase could not be demonstrated. Thus, the method for obtaining synchrony described here corresponds to one additional phase of synchrony (block in S-phase, synchrony lasts for the G2-phase).

Fuerst et al. (1996) described partially synchronised Arabidopsis cell suspension cultures with a peak mitotic index of 9% with a broad spread over more than 12 hours, which was achieved after arresting the cell with low concentration of cycloheximide and release by washing. At the concentration used, cycloheximide is reported to inhibit a G1-specific step, but not general protein synthesis. In this work, a population of laggard cells resulted in significant overlap between the S-phase and mitotic peaks, indicating that synchrony was substantially lost before the end of the S-phase.

Riou-Khamlichi et al. (1999) disclose results using sucrose removal and resupply that show synchrony during G1 phase and entry into S phase, but the further continuation of synchrony is not evident or reported.

It is clear from the publications mentioned above that the art is deficient in providing Arabidopsis thaliana cell suspension cultures that can be synchronised to a sufficiently high level, and particularly have the capacity of remaining synchronised for a sufficient time period. No methods are described to date that provide even partial synchrony of Arabidopsis cell suspension cultures for more than two complete sequential cell cycle phases. None of the described methods yield a complete (partially) synchronous cell cycle. Such a complete synchronised cell cycle is required for the systematic analysis of different cell cycle transitions using an Arabidopsis cell suspension culture, and consequently no methods for such analysis have been described. Furthermore, no set of methods, using various blocking procedures for a single Arabidopsis cell culture that allows investigation of transition between different cell cycle phases has been made available in the art.

The present invention provides a solution to this problem as described in the different embodiments, summarised below, and described to a further extent in the detailed description and examples as well as in the claims.

### BRIEF DESCRIPTION OF THE FIGURES

FIG. 1: Graphical representation of the labelling index (LI) and mitotic index (MI) in function of time, in the synchronised Arabidopsis cell suspension cultures MM1 and MM2d, treated as described in the Examples. —□—: LI for MM1—●—:LI for MM2d —∘—:MI for MM1—Δ—:MI for MM2d.

### SUMMARY OF THE INVENTION

The invention provides a method for producing a synchronised Arabidopsis cell suspension culture comprising the steps of:

a) arresting a suitable number of cells of an Arabidopsis cell suspension culture in a distinct stage of the cell cycle by a reversible block;

b) removing the reversible block to allow the arrested cells to progress through the cell cycle; and

c) further culturing the Arabidopsis cell suspension culture under suitable conditions;

wherein the Arabidopsis cell suspension culture is capable of an increase in cell number of at least 8 fold within 7 days, preferably of an increase of at least 15 fold within 7 days, particularly at least 20 fold within 5 days; or the Arabidopsis cell suspension culture has a cell doubling time of less than about 30 hrs, preferably less than about 25 hrs, particularly has a cell doubling time of about 24 hrs. Preferably, the Arabidopsis culture is capable of producing chlorophyll under suitable light conditions, particularly the Arabidopsis cell suspension culture is MM1 (DSM 13563) or MM2d (DSM13564) or a derivative thereof, particularly a transgenic derivative thereof. Preferably, the Arabidopsis cell suspension culture is diluted before the blocking step, particularly the early stationary phase Arabidopsis cell suspension culture is diluted about two to sevenfold, particularly about five-fold.

The invention also provides a method for producing a synchronised Arabidopsis cell suspension culture comprising the steps of:

a.) contacting an Arabidopsis cell suspension culture with a first cell cycle blocking compound, preferably selected from the group of aphidicolin, propyzamide, roscovitine, olomoucine, mimosine, quercitine, and abscisic acid, particularly propyzamide or aphidicolin or roscovitine, in a concentration and for a period of time sufficient to arrest a suitable number of cells of the cell suspension culture in a distinct stage of the cell cycle, preferably in a concentration of about 0.2 μg/ml to about 170 μg/ml, particularly in a concentration of about 4 μg/ml;

b.) removing the cell cycle blocking compound from the cell suspension culture after the period of time to arrest a suitable number of cells of the cell suspension culture to produce a synchronised culture; and

c.) incubating the synchronised culture under suitable culture conditions; wherein the Arabidopsis cell suspension culture is capable of an increase in cell number of at least 8 fold within 7 days, preferably of an increase of at least 15 fold within 7 days, particularly at least 20 fold within 5 days or the Arabidopsis cell suspension culture has a cell doubling time of less than about 30 hrs, preferably less than about 25 hrs, particularly has a cell doubling time of about 24 hrs. Preferably, the Arabidopsis culture is capable of producing chlorophyll under suitable light conditions, particularly the Arabidopsis cell suspension culture is MM1 (DSM13563) or MM2d (DSM13564) or a derivative thereof, particularly a transgenic derivative thereof.

It is another object of the invention to provide a method for producing a synchronised Arabidopsis cell suspension culture comprising the steps of

a.) contacting an Arabidopsis cell suspension culture capable of an increase in cell number of at least 8 fQld within 7 days, preferably of an increase of at least 15 fold within 7 days, particularly at least 20 fold within 5 days, or an Arabidopsis cell suspension culture having a cell doubling time of less than about 30 hrs, preferably less than about 25 hrs, particularly having a cell doubling time of about 24 hrs, more particularly an Arabidopsis culture which is capable of producing chlorophyll under suitable light conditions, especially the Arabidopsis cell suspension culture is MM1 (DSM13563) or MM2d (DSM13564) or a derivative, particularly a transgenic derivative thereof, with a first cell cycle blocking compound, preferably selected from the group of aphidicolin, propyzamide, roscovitine, olomoucine, mimosine, quercitine, cycloheximide or abscisic acid, particularly propyzamide, or aphidicolin, or roscovitine, or cycloheximide, in a concentration and for a period of time sufficient to arrest a suitable number of cells of the cell suspension culture in a distinct stage of the cell cycle, preferably in a concentration of about 0.2 μg/ml to about 170 μg/ml, particularly in a concentration of about 1 to about 20 μg/ml;

b.) removing the cell cycle blocking compound from the cell suspension culture after the period of time to produce a synchronised culture; and

c.) incubating the synchronised culture under suitable culture conditions;

d.) contacting the synchronised cell suspension culture with a second cell cycle blocking compound, preferably selected from the group of aphidicolin, propyzamide, roscovitine, olomoucine, mimosine, quercitine, and abscisic acid, particularly propyzamide, or aphidicolin, or roscovitine, in a concentration and for a period of time sufficient to arrest a suitable number of cells of cell suspension culture in a distinct stage of the cell cycle, preferably in a concentration of about 0.2 μg/ml to about 170 μg/ml, particularly in a concentration of about 1 to about 20 μg/ml;

e.) removing the cell cycle blocking compound from the cell suspension culture after the period of time to yield a synchronised culture; and

f.) incubating the synchronised culture under suitable culture conditions; wherein the second cell cycle blocking compound is capable of arresting cells of the cell suspension culture in another stage of the cell cycle than the stage wherein the cells are blocked by contacting with the first cell cycle blocking compound, preferably wherein the first cell cycle blocking compound is aphidicolin and the second cell cycle blocking compound is propyzamide.

The invention also provides a method for producing a synchronised Arabidopsis cell suspension culture comprising the steps of

a) incubating the Arabidopsis cell suspension culture in a culture medium lacking one or more of the culture medium compounds required for the growth of the suspension culture, preferably lacking at least a carbohydrate, a nitrate, a phosphate, an auxin or a cytokinin, particularly lacking at least sucrose, potassium nitrate, potassium dihydrogen phosphate, naphthalene acetic acid or kinetin, for a period of time sufficient to arrest a suitable number of cells of cell suspension culture in a distinct stage of the cell cycle;

b) replacing the medium lacking the compound required for the growth of the suspension culture with a medium comprising the previously lacking compound or a compound with similar function, or adding the required compound to the medium lacking the compound to produce a synchronised cell suspension culture;

c) incubating the synchronised culture under suitable culture conditions;

wherein the Arabidopsis cell suspension culture is capable of an increase in cell number of at least 8 fold within 7 days, preferably of an increase of at least 15 fold within 7 days, particularly at (east 20 fold within 5 days or the Arabidopsis cell suspension culture has a cell doubling time of less than about 30 hrs, preferably less than about 25 hrs, particularly has a cell doubling time of about 24 hrs. Preferably, the Arabidopsis culture is capable of producing chlorophyll under suitable light conditions, particularly the Arabidopsis cell suspension culture is MM1 (DSM13563) or MM2d (DSM13564) or a derivative thereof, particularly a transgenic derivative thereof and preferably, wherein the Arabidopsis cell suspension culture is diluted before the incubation, preferably wherein an exponentially growing or early stationary phase suspension culture is diluted about two to sevenfold, particularly about five-fold. Optionally, the method may be supplemented by the following further steps of contacting the synchronised cell suspension culture with a cell cycle blocking compound, preferably selected from the group of aphidicolin, propyzamide, roscovitine, olomoucine, mimosine, quercitine, abscisic acid, and cycloheximide particularly propyzamide or aphidicolin or roscovitine or cycloheximide in a concentration and for a period of time sufficient to arrest a suitable number of cells of cell suspension culture in a distinct stage of the cell cycle, preferably in a concentration of about 0.2 μg/ml to about 170 μg/ml, particularly in a concentration of about 1 to about 20 μg/ml; removing the cell cycle blocking compound from the cell suspension culture after the period of time to yield a synchronised culture; and incubating the synchronised culture under suitable culture conditions;

It is yet another object of the invention to provide a synchronised Arabidopsis cell suspension culture wherein said cell suspension culture has a maximum labelling index of at least 15% or a maximum mitotic index of at least 9% when cells of said suspension culture are progressing through the cell cycle, preferably obtainable by the methods of the invention.

The invention also provides a synchronised Arabidopsis cell suspension culture wherein synchrony is maintained for at least one additional cell cycle phase, preferably two additional cell cycle phases, particularly at least three cell cycle phases after the completion of the cell cycle phase in which the cells of the cells suspension culture are blocked, quite particularly wherein synchrony is maintained for a complete cell cycle, as observed by a detectable level of synchrony persisting after cells have passed through all 4 cell cycle phases, such as the presence of a second MI or LI peak, albeit smaller, of cells in S or M-phase.

It is another object of the invention to provide a synchronised Arabidopsis cell suspension culture wherein the labelling index has declined to less than one half, preferably one third of its peak value before the mitotic index has reached more than one half, preferably more than one third of its peak value. Most preferably, the labelling index should decline below 20% of the maximum LI value measured before the mitotic index increases to 20% of its respective maximum value.

The invention further relates to a method for analysing the timing of expression, preferably transcription, of a gene of interest during the cell cycle of an Arabidopsis cell in a suspension culture comprising the steps of:

a.) producing at a particular time point after the release from the cell cycle block a sample of a synchronised Arabidopsis cell suspension wherein said cell suspension culture has a maximum labelling index of at least 15% or a maximum mitotic index of at least 9% when cells of said suspension culture are progressing through the cell cycle, preferably obtained through the methods of the invention;

b.) extracting RNA, preferably polyadenylated RNA from the sample;

c.) identifying the relative level of an RNA molecule hybridising with a nucleotide sequence of at least 20 consecutive nucleotides, preferably at least 100 consecutive nucleotides of said gene of interest.

In addition, the invention relates to a method for identifying or analysing a protein whose abundance, state of modification or enzymatic activity varies during the cell cycle comprising the steps of

a) producing at a particular time point after the release from the cell cycle block a sample of a synchronised Arabidopsis cell suspension culture wherein said cell suspension culture has a maximum labelling index of at least 15% or a maximum mitotic index of at least 9% when cells of said suspension culture are progressing through the cell cycle, preferably obtained through the methods of the invention;

b) extracting proteins from the sample of cells;

c) identifying the relative level, state of modification or enzymatic activity of proteins in cells of the sample;

d) optionally, producing samples at other time points and reiterating steps a to c.

The invention also provides a method for analysing the relative level of a particular metabolite during the cell cycle of an Arabidopsis cell comprising the steps of

a.) producing at a particular time point after the release from the cell cycle block a sample of a synchronised Arabidopsis cell suspension culture wherein said cell suspension culture has a maximum labelling index of at least 15% or a maximum mitotic index of at least 9% when cells of said suspension culture are progressing through the cell cycle, preferably obtained through the methods of the invention;

b.) analysing the relative level of a particular metabolite in cells of the sample.

The invention also relates to a method for isolating a cell cycle regulated gene or a corresponding cDNA comprising the steps of

a.) producing a first sample of a synchronised Arabidopsis cell suspension culture wherein said cell suspension culture has a maximum labelling index of at least 15% or a maximum mitotic index of at least 9% when cells of said suspension culture are progressing through the cell cycle, preferably obtained through the methods of the invention;

b.) producing a second sample of a synchronised Arabidopsis cell suspension culture wherein said cell suspension culture has a maximum labelling index of at least 15% or a maximum mitotic index of at least 9% when cells of said suspension culture are progressing through the cell cycle, preferably obtained through the methods of the invention, the second sample differing from the first sample in at least one condition;

c.) extracting RNA, preferably polyadenylated RNA from the first and the second sample;

d.) identifying an RNA molecule with a different concentration in cells of the first sample when compared to cells of the second sample; and

e.) isolating a gene or cDNA corresponding to the identified RNA molecule

Optionally a transgenic plant comprising the isolated cDNA or cell cycle regulated gene may be produced.

### DETAILED DESCRIPTION OF PREFERRED EMBODIMENTS OF THE INVENTION

The availability of Arabidopsis thaliana cell suspension cultures that can be synchronised to a sufficiently high level and remain synchronised for a period of time allowing inter alia the identification and isolation of genes expressed in particular phases of the cell cycle, preferably expressed selectively in specific phases of the cell cycle, particularly expressed selectively in the G1, S, G2 or M phase of the cell cycle, or at boundaries or control points between and within these phases or during the transition from resting, quiescent or non-dividing cells (sometimes referred to as G0-phase cells) back into the cell division cycle could be of great advantage in the isolation and direct analysis of cell cycle regulated genes, particularly cell cycle genes in plants. It is clear that such synchronised cell suspension cultures from Arabidopsis thaliana would obviate the need of using cell suspension cultures from plants, which have the capacity to be synchronised to a high extent (such as tobacco BY-2 cell suspension) but which have other limitations. In particular, the use of synchronised Arabidopsis thaliana cell suspension cultures may allow direct identification and analysis of cell cycle regulated genes, proteins, enzyme activities or metabolites using the unique molecular tools available for this model plant species.

It is clear from the publications mentioned in the above background that the art is deficient in providing Arabidopsis thaliana cell suspension cultures that can be synchronised to a sufficiently high level, and particularly have the capacity of remaining synchronised for a sufficient time period.

The present invention provides a solution to this problem as described in the different embodiment and the claims. It is based on the observation that treatment of fast-growing Arabidopsis cell suspension cultures, preferably with a cell doubling time of about 24 hrs or less, capable of growth in small clusters of cells and which are preferably capable of synthesising chlorophyll under suitable light conditions, with at least one cell cycle blocking compound resulted in a high level of synchronicity in the cells of the suspension culture and that these cells remained predominantly synchronised for more than one cell cycle phase following the cell cycle phase in which the majority of the cells of the suspension culture have been blocked. Similar results were obtained by blocking such Arabidopsis suspension cultures, at low cell density, by omitting an essential compound required for growth of the cells from the incubation medium. Nowhere in the prior art has it been suggested that of all possible variables involved in optimising synchronisation of Arabidopsis cell suspension cultures, such as use of different Arabidopsis cell cultures or different synchronisation methods available in general for plant cell cultures, the combination of these particular cell suspension cultures with these specific treatments would be the key to successful synchronisation methods for Arabidopsis cell suspension cultures.

In one embodiment of the invention, a method is provided for producing a synchronised Arabidopsis cell suspension culture comprising the steps of:

a) arresting a suitable number of cells of a suitable Arabidopsis cell suspension culture, preferably a diluted early stationary phase cell suspension culture, in a distinct stage of the cell cycle by a reversible block;

b) removing the reversible block to allow the arrested cells to progress through the cell cycle; and

c) further culturing the Arabidopsis cell suspension culture under suitable conditions.

“Suitable Arabidopsis cell suspension cultures” as used herein, are fast-growing Arabidopsis suspension cultures wherein the cells are well dispersed, particularly Arabidopsis cell suspension cultures wherein the Arabidopsis cell suspension culture has a cell doubling time of less than about 30 hrs, preferably less than about 25 hrs, particularly has a cell doubling time of about 24 hrs. Expressed otherwise, particularly suited Arabidopsis cell suspension cultures are those cultures which are capable of an increase in cell number of at least 8 fold within 7 days, preferably of an increase of at least 15 fold within 7 days, particularly at least 20 fold within 5 days. Preferred conditions to grow the cells for determining the above parameters are as exemplified in the detailed Examples.

Preferably, the Arabidopsis culture is capable of producing chlorophyll under suitable light conditions. “Suitable light conditions” inducing the synthesis of chlorophyll in plant cell suspensions are known to the skilled artisan. Preferably, the cell suspension is kept under continuous light conditions, particularly under a light intensity of approximately 1300 Lux (measured at the distance from the light source defined by the surface of the culture medium within the flasks) using cold light emitted from lamps with similar specifications as TLD HF 50W/840 produced by Philips (NL). It is emphasised that the procedures described here may be carried out on such cells whether cultivated in the dark or under suitable light conditions.

Particularly suitable Arabidopsis cell suspension cultures are those cell suspension cultures which are derived from the suspension culture of Arabidopsis thaliana ecotype Landsberg erecta described by May et al. 1993, such as the cell suspension culture MM1 which has been deposited under DSM number13563 and which is green under suitable light conditions, as well as cell suspension cultures derived thereof, including the cell suspension culture MM2d which has been deposited under DSM number 13564 and which has been derived from the previously mentioned culture by continuing cultivation in the dark and selection for faster growing cell suspension cultures.

It goes without saying that a cell suspension culture derived from the mentioned Arabidopsis cell suspension cultures includes transgenic cell suspension cultures, wherein at least one foreign DNA has been introduced into the genome, preferably the nuclear genome, of the plant cells, particularly wherein at least one foreign DNA has been stably integrated into the genome of the cells of the Arabidopsis cell suspension culture.

As used herein “foreign DNA” refers to a DNA sequence which is not in the same genomic environment in a transgenic plant cell wherein such DNA has been introduced, as is such a DNA when it is naturally found in a cell of the plant, bacteria, animal, fungus, virus or the like, from which such a DNA originates. Introduction of foreign DNA thus comprises but is not limited to introduction of chimeric genes wherein the DNA of interest is not operably linked to e.g. the same promoter and/or 3′ end, but may also comprise introduction of additional copies of DNA, particularly genes, derived from similar plant cells, as well as introducing an (allelic) variation of an endogenous DNA sequence, preferably endogenous gene, or replacement of an endogenous DNA sequence, preferably endogenous gene by such a variant DNA sequence.

Methods to introduce DNA into plant cells or to stably integrate DNA into the genome of plant cells are available in the art and include, but are not limited to, Agrobacterium-mediated transformation (Komari, 1989) or direct DNA transfer using PEG-mediated DNA introduction (Lee et al. 1988) electroporation (see e.g. US patent U.S. Pat. No. 5,679,558) or microprojectile bombardment mediated transformation (see e.g. a review by Songstad et al., 1995 and references therein).

Conveniently, the cells may be blocked by addition of chemical compounds to a suitable Arabidopsis cell suspension culture, but may also be blocked in any other way such as e.g. by omission of an essential compound in the culture medium required for the growth of the cells.

In a preferred embodiment of the invention, a method is provided for producing a synchronised Arabidopsis cell suspension culture, comprising the following steps:

a) a suitable Arabidopsis cell suspension culture is contacted with a cell cycle blocking compound, in a concentration and for a period of time sufficient to arrest a suitable number of cells of the cell suspension culture in a distinct stage of the cell cycle;

b) after the mentioned period of time, the cell cycle blocking compound is then removed from the cell suspension culture to produce a synchronised culture; and

c) the synchronised culture is further incubated under suitable culture conditions.

As used herein, a “cell cycle blocking compound” is a compound which when added to a cell culture, preferably a cell suspension culture, results in a reversible inhibition of the progression of the cell cycle for a majority of the cells of the cell suspension culture at a particular stage of that cell cycle. Preferably the cells of the suspension culture are arrested before mitosis or in late G1/early S phase.

Particularly suitable cell cycle blocking compounds for the methods of the invention are aphidicolin, propyzamide, roscovitine, olomoucine, mimosine, quercitine, abscisic acid, or cycloheximide, particularly propyzamide or aphidicolin or roscovitine or cycloheximide. Table 1 summarises the target for the inhibitory activity of these different cell cycle blocking compounds, as well as the different phases of the cell cycle at which cells are arrested pursuant to addition of the compound.

| Compound | target | cell cycle arrest |
|---|---|---|
| aphidicolin | DNA polymerase alpha | G1/early S |
| propyzamide | anti tubulin | M |
| roscovitine | cyclin dependent kinase activity | G1/S or G2/M |
| olomoucine | cyclin dependent kinase activity | G1/S or G2/M |
| mimosine | DNA replication | early S |
| cycloheximide | protein synthesis inhibitor | G1 |
| quercitin | broad range of enzymes | G1 |
| abscisic acid | plant hormone arresting division | G1 |

Other suitable cell cycle blocking compounds may be bohemine or the ribonucleotide reductase inhibitor hydroxyurea, the latter blocking at the G1/S phase boundary.

It is expected that it may advantageous to use more than one compound blocking cells in the same phase of the cell cycle at lower concentrations than if such compound were to be used alone.

The optimal concentration of cell cycle blocking compound in the medium may vary depending on the specific compound used, and will depend on its strength of inhibition (as measured by its Ki). For aphidocolin, for example, it was found that approximately 4 μg/ml is a suitable concentration, but it is expected that concentrations of a cell cycle blocking compound up to about 170 μg/ml or as low as about 0.2 μg/ml can be used to good effect, depending on the particular cell cycle blocking compound used. For propyzamide, for example, it was found that approximately 1 to 2 μg/ml, particularly 1.6 μg/ml is a suitable concentration, but again, it is expected that concentrations up to 20 μg/ml or as low as 0.1 μg/ml can be used to achieve a similar effect. Similar concentrations apply to other cell cycle blocking compounds.

The period of time of contact between the cell cycle blocking compound and the cells may also vary depending on a number of parameters, such as the specific compound used, the strength of inhibition of the compound, the concentration of the compound used etc. However, it is believed that the cell cycle blocking compound should not be left in contact with the cultured cells for more than approximately 48 hours. For aphidicolin, particularly in a concentration of about 4 μg/ml, an optimal period of time is about 24 hrs, but it is expected that a contacting time of about 2 hrs may be sufficient for the purposes of the invention. For propyzamide, particularly in a concentration of about 1.6 μg/ml, an optimal period of time is about 6 hrs, but it is expected that a contacting time of about 0.5 hrs may be sufficient for the purposes of the invention.

A person skilled in the art may determine the mentioned sufficient period of time by determining the percentage of cells which have been arrested in their progression through the cell cycle. Ideally at least about 90%, particularly almost all of the cells should be blocked, however, the methods of the invention can be carried out when at least about 40% of the cells, preferably at least about 50%, particularly at least about 60%, more particularly at least about 75% of the cells are blocked in a particular cell cycle phase. Moreover, the technical feasibility of blocking cells may be limited by the particular of cell cycle blocking compound used, particularly by the cell cycle phase in which the cells are blocked.

After the cell cycle blocking compound has been contacted with the cultured cells, for a period of time, the cell cycle blocking compound is removed. This removal can be conveniently done e.g. by separating the cultured cells from the medium, washing the cells, and incubating them in new culture medium without the cell cycle blocking compound. The relief from the inhibition by the cell cycle blocking compound, allows the cells in the cell suspension to reenter in the cell cycle. The degree of synchrony in the released cell population can be conveniently determined by identifying the labelling index (LI) and mitotic index (MI) over a period of up to 24 hrs. In one embodiment of the invention, methods are provided to produce synchronised cell cultures reaching at least at LI of about 15% or a MI of about 9%, more preferably a LI of 40% or a MI of 15%, most preferably a LI of more than 70% or a MI of more than 20%.

In another embodiment of the invention a method is provided for producing a synchronised Arabidopsis cell suspension culture, comprising the following steps:

a) a suitable Arabidopsis cell suspension culture as defined elsewhere in this application is contacted with a first cell cycle blocking compound, in a concentration and for a period of time sufficient to arrest the cells of the cell suspension culture in a distinct stage of the cell cycle;

b) after the mentioned period of time, the cell cycle blocking compound is then removed from the cell suspension culture to produce a synchronised culture; and

c) the synchronised culture is incubated under suitable culture conditions;

d) the synchronised cell suspension culture is then incubated with a second cell cycle blocking compound in a concentration and for a period of time sufficient to arrest the cells of said cell suspension culture in a distinct stage of the cell cycle different than the stage wherein said cells are blocked by contacting with the first cell cycle blocking compound;

e) removing said cell cycle blocking compound from said cell suspension culture after said period of time to yield a synchronised culture; and

f) incubating said synchronised culture under suitable culture conditions; wherein said second cell cycle blocking compound is capable of arresting cells of said cell suspension culture in another stage of the cell cycle than said stage wherein said cells are blocked by contacting with said first cell cycle blocking compound.

A preferred combination of first and second cell cycle blocking compounds is aphidicolin and propyzamide. Alternative combinations may be aphidicolin and roscovitine, aphidicolin and olomoucine, roscovitine and propyzamide, olomoucine and propyzamide, mimosine and propyzamide, cycloheximide and roscovitine, cycloheximide and olomoucine, or cycloheximide and propyzamide.

Preferred embodiments for concentration of cell cycle blocking compounds, period of time of contact etc. are as described for synchronisation methods comprising at least one step of treating with a cell cycle blocking compound.

Having read this description, the person skilled in the art will immediately realise that the methods of the invention for synchronising Arabidopsis cell suspension cultures using cell cycle blocking compounds may be further combined with a step of synchronising the cell suspension culture by blocking cell division of the cells of the cell suspension culture through starvation for an essential compound, such as phosphates, sucrose or hormones, as described e.g. in Riou-Kamlichi et al. (1999), followed by readdition of the lacking compound to further improve the quality of the synchronisation.

The additional process steps of blocking the cell division of the cells from the cell suspension culture by starvation for an essential compound, and release of the cell division block, may be performed before or after the step of treating with a cell cycle blocking compound, or even in-between the steps of treating with a cell cycle compound in the case where more than one treatment with such a cell cycle blocking compound is performed.

In another embodiment of the invention a method for producing a synchronised Arabidopsis cell suspension culture is provided which comprises the steps of

a) incubating a suitable Arabidopsis cell suspension culture in a culture medium lacking one or more of the culture medium compounds required for the growth of the suspension culture, preferably lacking at least a carbohydrate, a nitrate, a phosphate, an auxin or a cytokinin, particularly lacking at least sucrose, potassium nitrate, potassium dihydrogen phosphate, naphthalene acetic acid or kinetin, for a period of time sufficient to arrest a suitable number of cells of cell suspension culture in a distinct stage of the cell cycle;

b) replacing the medium lacking the compound required for the growth of the suspension culture with a medium comprising the previously lacking compound or a compound with similar function, or adding the required compound to the medium lacking the compound to produce a synchronised cell suspension culture;

c) incubating the synchronised culture under suitable culture conditions;

It has been found that in contrast to the methods available in the art for synchronising Arabidopsis suspension cultures through starvation for an essential culture medium component, the use of the Arabidopsis cultures as defined provides unexpected better synchrony, particularly when using the culture MM2d (DSM 13564). Moreover the Arabidopsis cell suspension culture should preferably be diluted before the incubation, particularly about two to sevenfold, quite particularly about five-fold. Furthermore, it is preferred to use cells from suspension cultures in early stationary phase or mid-exponential phase (particularly for MM2d cells).

The invention also relates to synchronised cell suspension cultures, obtainable by the synchronisation methods of the invention. As used herein, a “synchronised cell suspension culture” refers to a cell suspension culture wherein at a particular time point after the cell cycle blocking compound has been removed, a certain part of the suspended cells are in the same phase in the progression through the cell cycle. The methods of the invention are particularly suited to produce synchronised cell suspension cultures, with a synchrony such that a maximum labelling index of at least 15%, preferably at least 20%, particularly at least 50%, more particularly about 70% or a maximum mitotic index of at least 9%, preferably at least 15%, particularly at least about 20% would be obtained if the Arabidopsis cell suspension were allowed to progress. In other words, the synchronous Arabidopsis cell suspension cultures of the invention exhibit a more than two fold increase, preferably a more than 3 fold increase in labelling or mitotic index over the level found in the corresponding asynchronous exponential cell suspension culture. One possible way of determining the degree of synchronicity is to determine the number of cells from a cell suspension culture that are in a particular phase of the cell cycle, preferably a cell cycle phase which can be easily recognised, such as but not limited to mitosis (“mitotic index”) or S-phase (“labelling index”). It is clear that synchronicity of a given cell suspension culture is not only determined quantitatively by the largest number of cells present in a particular phase (“peak height”), but also qualitatively by the time period during which a considerably amount of cells from the cell culture is in the particular phase (“peak width”). The peak width may be measured as the time interval across the peak at a position equivalent to half its maximal value. It will be clear however that the peak width for a given cell cycle phase depends both on the length it takes one cell to traverse the phase, as well as the degree of synchrony between the cells. Using the methods of the invention, synchronised cultures may be obtained having a peak widths for mitotic or labelling index of less than about 10 hrs, preferably having a peak width of less than about 7.5 hrs, particularly having a peak width of about 5 hrs. S-phase peak width typically may be about 3 hr; M-phase width typically may be about 3 to 7 hr.

A mitotic index may be calculated by staining the cells with e.g. a Hoechst stain No 33258 (2′-[4-Hydroxyphenyl]-5-[4-methyl-1-piperazinyl]-2,5′-bi-1 H-benzimidazolel or DAPI (4,6-Diamidino-2-phenylindole) and estimating the percentage of cells with mitotic figures with a microscope under UV-light (Planchais et al, 1997). A labelling index may be calculated by estimating the percentage of cells incorporating bromodeoxyuridine in their replicating DNA as revealed by antibody detection (Miyake et al., 1997, Mader and Hanke, 1996; Levi et al., 1987; Gratzner et al., 1975)

The level of synchronicity may also be estimated by analysing the DNA content in the cells of the cell suspension culture, e.g. via flow cytometry using an instrument such as Partec PAS-III or other suitable instrument and appropriate analysis software such as MULTICYCLE (Reichfeld et al., 1999; Glab et al., 1994).

The level of synchronicity may also be estimated by analysing the expression levels of known cell-cycle regulated genes, (such as HISTONE H4, CYCLINB1, CYCLIND3, CYCLIND2 or CDC2b encoding genes) in a cell suspension at various times after removing the cell cycle blocking compound.

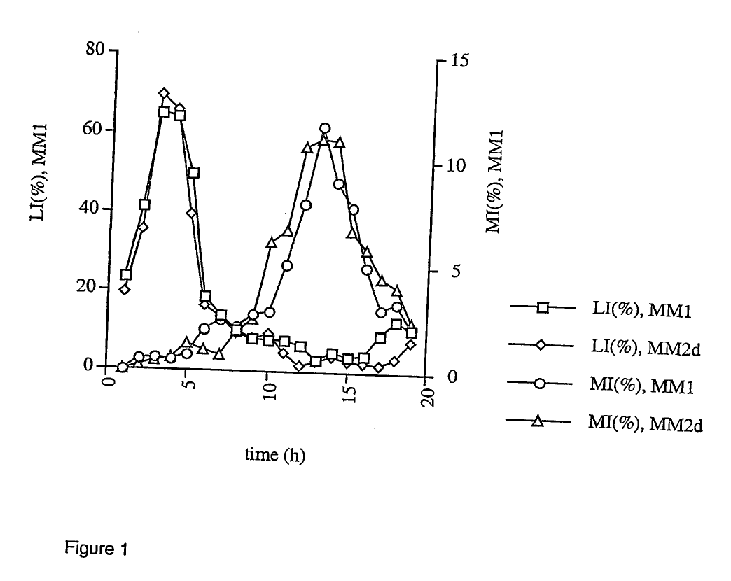

The synchronised Arabidopsis cell suspension cultures obtainable by the methods of the invention are also distinguished from the synchronised cultures available in the art, by their ability to maintain synchrony for at least one additional cell cycle phase, preferably two additional cell cycle phases, particularly at least three cell cycle phases after the completion of the cell cycle phase in which the cells of the cells suspension culture are blocked. Quite particularly, the methods of the invention allow to obtain Arabidopsis cell suspension cultures wherein synchrony is maintained for a complete cell cycle. A further distinction with the synchronised Arabidopsis cell suspension cultures of the art is that the synchronised Arabidopsis cell suspension culture obtainable by the methods of the invention have a very distinct phase wherein the majority of the cells are in the S-phase (evidenced by labelling index and flow cytometry analysis) well separated form the phase wherein the majority of the cells are in the M-phase (evidenced by mitotic index). In other words, the methods of the invention allow to obtain synchronised Arabidopsis cell suspension cultures wherein the labelling index has declined to less than one half, preferably one third of its peak value before the mitotic index has reached more than one half, preferably more than one third of its peak value (see FIG. 1).

The invention further provides methods for isolating cell cycle regulated genes or cDNAs corresponding to cell cycle regulating genes comprising the following steps:

a.) A first sample is produced of a synchronised Arabidopsis cell suspension culture wherein said cell suspension culture has a maximum labelling index of at least 15% preferably at least 20%, particularly at least 50%, more particularly about 70% or a maximum mitotic index of at least 9%, preferably at least 15%, particularly at least about 20% when cells of said suspension culture are progressing through the cell cycle. Preferably, the synchronised cell suspension culture is produced according to the methods of the invention.

b.) A second sample of a synchronised Arabidopsis cell suspension culture wherein said cell suspension culture has a maximum labelling index of at least 15% preferably at least 20%, particularly at least 50%, more particularly about 70% or a maximum mitotic index of at least 9%, preferably at least 15%, particularly at least about 20% when cells of said suspension culture are progressing through the cell cycle, differing from said first sample in at least one condition, is produced. The different condition between the first and the second sample may be the difference in time period between the time of release from the reversible block and the time of sampling. The first and second sample may also be taken from different synchronised cell suspension cultures, at similar time points, whereby the different condition may be e.g. the different genetic background or different environmental conditions. The first and second sample may also be taken from different synchronised cell suspension cultures at different time points.

c.) RNA, preferably polyadenylated RNA, is extracted from the first and second sample according to methods known in the art.

d.) Using techniques established in the art, including RNA fingerprint technology (see e.g. publication of the PCT application WO 9641011) or differential display technology, such as the READSTM technology (see US patent U.S. Pat. No. 5,712,126) RNA molecules with a different concentration in cells of the first sample when compared to cells of the second sample, are identified. It goes without saying that the person skilled in the art will realise that other available methods for determining differences in expression levels, particularly transcripton levels between samples may be used.

e.) The cDNA or gene(s) corresponding to the identified RNA molecule(s) are isolated.

f.) Optionally, the isolated cDNA(s) or gene(s) may be used to generate transgenic plants comprising such cDNA(s) or gene(s), whereby the cDNAs may be expressed using heterologous promoters and transcription/termination signals. Such genes or cDNAs may be used to modulate plant characteristics including growth, development, form, vigour, yield, size of different organs such as the endosperm, seed or other portion of the plant, or to modulate cellular processes such as cell division or endoreduplication.

It is clear that the described methods for identifying and isolating cell cycle regulated genes also includes variations wherein particular mRNA levels of more than two samples are compared to find differences under different conditions.

The present invention also relates to DNA sequences encoding a cell cycle regulated protein or a cell cycle protein obtainable by the methods of the invention.

As used herein a “cell cycle regulated gene” is a gene of which the RNA expression level is specifically induced or down regulated during a certain phase in the cell cycle and which can serve as a marker gene to follow cell cycle progression. As used herein, a “cell cycle gene” is a gene that has a role in the control of cell cycle progression.

Once the cell cycle regulated genes, preferably cell cycle genes or cDNAs corresponding to such genes have been isolated, (and more particularly their DNA sequence has been determined), their function may be determined e.g. by generating plant cells or plants wherein the corresponding gene has been mutated (structural knock-out) and observing the phenotype of such cells or plants. The genes or cDNAs or their nucleotide sequence may also be used to generate plant cells or plants wherein the expression of the corresponding gene has been modulated, e.g. by expression of so-called anti-sense and/or co-suppressing genes (e.g. according to methods described in WO 99/53050)

The isolated cell cycle regulated genes, preferably cell cycle genes or cDNAs corresponding to such genes may also be used to generate transgenic Arabidopsis cell suspension cultures, suitable for the synchronisation methods described herein. Such transgenic Arabidopsis cell suspension cultures may then be used in comparison with e.g. the corresponding untransformed Arabidopsis cell suspension cultures for isolating further cell cycle regulated genes according to the methods described herein.

The synchronised Arabidopsis suspension cultures obtainable by the methods of the invention may also be used to analyse the timing of expression, preferably of transcription of a gene or nucleic acid of interest during the cell cycle of an Arabidopsis cell, to analyse variation in the abundance, state of modification or enzymatic activity of a given protein during the cell cycle and to identify and isolate genes influencing such abundance, state of modification or enzymatic activity of a given protein. Further, the synchronised Arabidopsis cell suspension cultures may also be used to analyse fluctuations in particular metabolites during the cell cycle and to identify and isolate genes influencing such fluctuations in particular metabolites. The synchronised Arabidopsis cell suspension cultures may also be used to isolate RNA at particular time points after release of the cell cycle block, and to construct cDNA libraries which are enriched, particularly which are selective for RNAs expressed, or expressed at higher or lower level during a particular phase in the cell cycle. Such cell cycle stage specific libraries may be used for conventional screening, but may also be used for more specific purposes such as e.g. two hybrid interaction screening or metabolic profiling. The methods for further processing the compounds isolated from the synchronised cell suspension cultures are well known to the person skilled in the art and have been well documented in the literature (e.g. Trethewey et al. 1999; Bartel et al. 1997)

The following examples describe the methods of the invention in detail. Unless stated otherwise in the Examples, all recombinant DNA techniques are carried out according to standard protocols as described in Sambrook et al. (1989) Molecular Cloning: A Laboratory Manual, Second Edition, Cold Spring Harbor Laboratory Press, NY and in Volumes 1 and 2 of Ausubel et al. (1994) Current Protocols in Molecular Biology, is Current Protocols, USA. Standard materials and methods for plant molecular work are described in Plant Molecular Biology Labfax (1993) by R. D. D. Croy, jointly published by BIOS Scientific Publications Ltd (UK) and Blackwell Scientific Publications, UK.

Arabidopsis suspension cultures MM1 and MM2d have been deposited by the Institute of Biotechnology, University of Cambridge (Tennis Court Road, Cambridge CB2 IQT, United Kingdom), under the Budapest Treaty at DSMZ (Deutsche Sammlung von Mikroorganismen und Zelikulturen GmbH; Mascheroder Weg 1b, D-38124 Braunschweig, Federal Republic of Germany) on Feb. 2, 2000 with the following deposit numbers:

Suspension culture MM1: DSM 13563

Suspension culture MM2d: DSM 13564

The depositor has authorized the applicant to refer to the deposited biological material in the application and has given his unreserved and irrevocable consent to the deposited material being made available to the public in accordance with Rule 28 of the European Patent Convention.

### EXAMPLES

### Example 1

### Culture Growth Determination

Cell suspension culture of Arabidopsis cell culture MM1 was maintained by sub-culturing every 7 days 10 ml into 200 ml fresh MSS-medium (Murashige and Skoog medium [without sucrose, without IAA, without kinetin, without agar, from ICN Biomedicals, Aurora, Ohio, USA, cat. no. 26-100-24], with added 3% w/v sucrose, 0.5 mg/l NAA, 0.05 mg/l kinetin) in 500 ml narrow necked Erlenmeyer flasks covered with 2 layers of domestic heavy duty aluminium foil with a piece of PVC film loosely laid over the top. MM1 was grown under continuous light conditions at approximately 1300 Lux from Philips TLD HF 50W/840 fluorescent tubes at 23-25° C. and rotated at 120 rpm on a New Brunswick (Edison, N.J., USA) G10 Gyrator shaker with 25 mm orbit. MM2d was maintained by sub-culturing every 7 days 5 ml of culture into 100 ml fresh MSS-medium in 300 ml narrow necked Erlenmeyer flasks and grown in continuous darkness and incubated at 27° C. and rotated at 130 rpm in a darkened New Brunswick Innova Model 4230 incubator shaker with 19 mm orbit.

5 ml of a MM1 and MM2d cultures, 7 days after previous subculture as described above, were inoculated into 100 ml MSS-medium and incubated. 1 ml samples were removed from both flasks at approximately 24 hr intervals, vigorously pipetted several times through a 1 ml Gilson pipette tip, and cells were counted using a haemocytometer (counting chamber 0.2 mm depth).

The following results were obtained:

| | MM1 | Cell doubling | | | |
|---|---|---|---|---|---|
| day | cell no. (x10\hat{ }5)/ml | time (h) | | | |
| 0 | 3.15 | | | | |
| 1 | 3.65 | 23.8 | | | |
| 2 | 7.35 | | 19.4 | | |
| 3 | 17.3 | | | 23.6 | |
| 4 | 35 | | | | 25.6 |
| 5 | 67 | | | | |
| 6 | 73.5 | | | | |
| 7 | 70 | | | | |
| 8 | 69 | | | | |

| | | |
|---|---|---|
| | MM2d | Cell doubling |
| day | cell no. (x10\hat{ }5) | time (h) |

| | | | | | |
|---|---|---|---|---|---|
| 0 | 3.2 | | | | |
| 1 | 3.42 | 15.5 | | | |
| 2 | 10 | | 17.4 | | |
| 3 | 26 | | | 20.6 | |
| 4 | 58.3 | | | | 22.5 |
| 5 | 122 | | | | |
| 6 | 108 | | | | |
| 7 | 126 | | | | |
| 8 | 113 | | | | |

Calculation of doubling time is based on the Monod model.

### Example 2

### Synchronisation of Arabidopsis Cell Suspension Cultures by Treatment with Aphidicolin

A. Synchronisation of MM1 by Treatment with Aphidicolin

Arabidopsis cell suspension culture MM1 (DSM 13563) was used for synchronisation experiments. The suspension culture was maintained by sub-culturing weekly 10 ml into 200 ml of fresh MSS-medium (MS-salt, 3% sucrose, 0.5 mg/l NAA, 0.05 mg/l kinetin) which is cultured in 500 ml narrow neck Erlenmeyer flasks, of which the opening is covered with double layer of aluminium foil and loosely covered with Saran wrap and which are placed on a GYROTORY® Shaker (New Brunswick Scientific). The cell suspension culture was grown under continuous light conditions (24 hrs a day); Philips lamps were used with the specification TLD HF 50W/840; light level is in average 1300 Lux and the cell suspension culture is rotated at 120 rpm at a temperature varying between 23-25° C. To arrest cells reversibly in late G1/early S-phase, cells were blocked with aphidicolin according to Nagata et al. 1992 (with slight modifications), 40 ml of an early stationary phase cell suspension culture was subcultured into 200 ml MSS-medium, 4.16 μg/ml aphidicolin was added to block cells at the end of G1/early S-phase, and the culture was incubated at 23° C., 120 rpm in the conditions as described above for MM1, for a period of 21.5 hrs. To remove aphidicolin, cells were washed through a nylon mesh (mesh size 47 μm) with 11 MSS-medium by vigorously shaking and resuspended in MSS-medium. The cells were vigorously shaken, for a washing time of about 14 min, and then centrifuged at 150Orpm for 1 min (387 rcf) with the brake switched off. The cell pellet was resuspended in 250 ml fresh MSS-medium, incubated under cultivation conditions as above and samples taken at various time points to determine the mitotic index (by DAPI staining) and labelling index (bromodeoxyuridine (BrdU) labelling) as well as for flow cytometry analysis.

Procedure to Determine Labelling Index (LI) and Mitotic Index (MI) to Follow Cell Cycle Progression.

To follow the transition of cells through S-phase after synchronisation using aphidicolin, labelling of cells with replicating DNA was carried out using bromo-deoxyuridine incorporation and antibody detection. The method which was used is according to Miyake et al., 1997 using the “RPN20 Amersham proliferation kit” with slight modifications to adjust the parameters to the Arabidopsis cell suspension culture. One hr before taking the appropriate sample for flow cytometry analysis, an aliquot of 5 ml suspension was transferred into a 100 ml Erlenmeyer flask and incubated with 10 μl labelling reagent for further 60 min. The cell suspension was spun down at 1500 rpm for 1 min (15 ml falcon tube) to harvest the cells, resuspended in PBS-buffer to wash the cells, and transferred into a 2 ml eppendorf tube. After and additional centrifuge step (2000 rpm/1 min), 1 ml of fixative (3.7% formaldehyde in PMEG; PMEG is 50 mM PIPES, 2 mM MgSO₄, 5 mM EGTA, 2% glycerol v/v, pH 6.8) was added and incubated at a rotary shaker (cold room) over night. After fixation a sample of the cells was washed 3 times with PBS-buffer (2000 rpm/min), prior enzyme-treatment (1% cellulase, 0.1% pectolyase, 0.4M mannitol) for 20 minutes at room temperature. After extraction with 1% Igepal CA-630 (Sigma) in PBS for 25 minutes at room temperature, the cells were washed twice with PBS-buffer, transferred to a slide (normal glass slide, coated with Vectabond™ reagent, Vector laboratories) and left to settle overnight in a humidified chamber. To block, the cells are incubated for 10 min with glycine (1%BSA, 0.05% Triton X-100, in PBS). The primary antibody mix (anti-5-bromo-2′-deoxyuridine and nuclease mix from Amersham proliferation kit, diluted 1:400) was added and incubated for 60-90 min at 30° C. in a humidified chamber (dark). The slides were washed carefully 3× with PBS for 5 min each (on slide).

A Texas-red-conjugated-donkey-antimouse (dil 1:400) (Jackson Immuno Research, Cat.No. 715-076-150) was used as secondary antibody allowing for binding for 60-90 min at 30° C. in a humidified chamber (dark). The cells were washed carefully 3× with PBS for 5 min each (on slide) before they were mounted in Vectashield containing DAPI (Cat.No. H-1200; Vector Laboratories). BrdU incorporation was visualised as bright red fluorescence by excitation with light of wavelength 575 nm. The labelling index was determined as the proportion of red fluorescent cells amongst the total number of cells observed using DAPI fluorescence. To determine the mitotic index of each sample, the fluorescence of DAPI under UV light excitation (350 nm) was used to determine the proportion of cells containing mitotic figures. In this experiment a LI peak of 65% and a MI peak of 12% were observed following release of cells from the aphidicolin block (see Table I and FIG. 1). Blocking the cells with aphidicolin revealed a MI peak of ranging from 10 to 15% and a labelling index of about 50-65%.

Procedure for Flow Cytometric Determination of Cell Cycle Phases

The level of synchronicity of the Arabidopsis cell suspension culture MM1 was analysed by flow cytometry. The cell pellet from 5 ml of cell suspension culture was frozen in liquid nitrogen. To analyse the DNA content, the High Resolution Kit for Plant ploidy analysis (Type P) (Partec, Cat. No. 06-5-4004) was used, containing solution A (to release cell nuclei) and solution B (to stain DNA with DAPI). A sample of the frozen cell pellet was transferred into a small petri-dish (5 cm diameter), 150 μl of solution A was added, and the sample was carefully chopped with vertical motion of a sharp razor blade. An additional 150 μl of solution A was added, the cell suspension pipetted up and down for several time before leaving for approximately 2 min at RT. The released cell nuclei were filtered through a 50 μm nylon mesh (Partec, CellTrics 50 μm, Cat. No. 06-4-2317) and 1 ml of solution B was added to the filtrate. On average 8200 particles were counted with a flow cytometer (PASIII; Partec GmbH, Germany), using HBO lamp excitation (Partec, mercury lamp HBO 100 long life, 100W) and detection of emission using a blue filter. Cell cycle phases were analysed using Multicycle for Windows (Phoenix Flow Systems, San Diego, Calif.). Flow cytometry results are not corrected for the mitotic cells (which are not measured by this method).

Table I shows data from analysis of samples obtained in the experiment described in Example 2A. Samples were collected at the time indicated (time of removal of aphidicolin equals time point 0 hrs) and analysed for the proportion of G1, S and G2 cells by flow cytometry, and for MI and LI as described in Example 2A. nd=not determined.

| | G1 (%) | | G2 (%) | Mitotic | Labelling |
|---|---|---|---|---|---|
| Time (h) | MM1 | S (%) MM1 | MM1 | index (%) | Index (%) |
| 0 | 50.6 | 30.1 | 19.3 | nd | nd |
| 1 | 23.7 | 64.9 | 11.4 | 0 | 23.21 |
| 2 | 23.8 | 64.8 | 11.4 | 0.5 | 41.3 |
| 3 | 27.2 | 62.5 | 10.4 | 0.58 | 65.06 |
| 4 | 23.2 | 32 | 44.8 | 0.5 | 64.13 |
| 5 | 19.9 | 16.3 | 63.8 | 0.68 | 50 |
| 6 | 16.1 | 0 | 83.9 | 1.88 | 18.43 |
| 7 | 13.8 | 0.5 | 85.7 | 2.37 | 13.67 |
| 8 | 10.9 | 0.3 | 88.8 | 2.04 | 10.32 |
| 9 | 11.1 | 0 | 88.9 | 2.63 | 8.1 |
| 10 | 10.8 | 0 | 89.2 | 2.84 | 7.86 |
| 11 | 13.4 | 0 | 86.6 | 5.06 | 7.65 |
| 12 | 15.1 | 0.8 | 84.1 | 7.96 | 6.8 |
| 13 | 22.9 | 0 | 77.1 | 11.67 | 3.08 |
| 14 | 31.5 | 0 | 68.5 | 9.03 | 5.05 |
| 15 | 38.6 | 0.2 | 61.3 | 7.86 | 3.68 |
| 16 | 33.4 | 6.9 | 59.6 | 5 | 4.09 |
| 17 | 36.3 | 6.2 | 57.5 | 2.96 | 9.46 |
| 18 | 35.7 | 8.2 | 56.1 | 3.24 | 12.96 |
| 19 | 38.4 | 6 | 55.6 | 2.06 | 10.91 |
| 20 | 41.1 | 10 | 48.8 | nd | nd |

B. Synchronisation of MM2d by Aphidicolin Treatment

This experiment was carried out as described for Example 1, except that cell line MM2d was used, and the culture conditions throughout the experiment were as described for MM2d in Example 1.

Synchronisation was performed as described in Example 2A, with the exception that before adding aphidicolin, 20 ml of the weekly subcultured cell suspension was transferred into 100 ml fresh MSS-medium in each of 2 separate flasks and cultivated under conditions as described in Example 1 for MM2d. After vigorously washing to remove aphidicolin, the cell pellet was resuspended in 250 ml MSS medium, divided between two 300 ml flasks and samples were taken at various time points to determine the mitotic index (DAPI) and labelling index (BrdU). Samples were taken at the same time frame for flow cytometry to follow cell cycle transition.

Samples were analysed by flow cytometry to determine the percentage of cells being in G1-, S- and G2M phase. The mitotic index (MI) and labelling index (LI) were independently determined (as described in Example 2A). Results are shown below in table II and in FIG. 1.

Table II. Aphidicolin block/release of Arabidopsis cell suspension MM2d. Data are shown from analysis of samples obtained in the experiment described in Example 2B. Samples were collected at the time indicated (time of removal of aphidicolin equals time point 0 hrs), and analysed for the proportion of G1, S and G2 cells, by flow cytometry, and for Ml and Li, using methods described in Example 2A.

| | G1 (%) | S (%) | G2 (%) | Mitotic | Labelling |
|---|---|---|---|---|---|
| Time (h) | MM2d | MM2d | MM2d | index (%) | Index (%) |
| 0 | 46.9 | 36.6 | 16.4 | nd | nd |
| 1 | 16.6 | 78.6 | 4.9 | 0 | 19.41 |
| 2 | 21.5 | 73.2 | 5.3 | 0.2 | 35.47 |
| 3 | 24.8 | 64.1 | 11.2 | 0.4 | 69.46 |
| 4 | 18.8 | 33 | 48.2 | 0.6 | 66 |
| 5 | 15.1 | 10.1 | 74.8 | 1.21 | 39.22 |
| 6 | 12.5 | 6.9 | 80.6 | 0.92 | 16.05 |
| 7 | 10.5 | 0 | 89.5 | 0.71 | 13.27 |
| 8 | 9 | 0 | 91 | 1.87 | 9.6 |
| 9 | 7.6 | 0 | 92.4 | 2.41 | 8.23 |
| 10 | 10.1 | 0 | 89.6 | 6.09 | 9.23 |
| 11 | 17.2 | 0 | 82.8 | 6.7 | 4.6 |
| 12 | 27.7 | 0 | 72.3 | 10.72 | 1.46 |
| 13 | 32.1 | 0 | 67.9 | 11.06 | 2.78 |
| 14 | 34.3 | 0 | 65.7 | 10.99 | 3.61 |
| 15 | 40.7 | 0 | 59.3 | 6.74 | 2.73 |
| 16 | 34.9 | 5.8 | 59.4 | 5.79 | 2.28 |
| 17 | 40.8 | 7.3 | 52 | 4.49 | 1.96 |
| 18 | 40.7 | 11.3 | 47.9 | 4.01 | 3.3 |
| 19 | 38.4 | 14.4 | 47.3 | 2.38 | 7.62 |
| 20 | 37.7 | 16.9 | 45.5 | nd | nd |

nd = not determined
Note that maximum S phase synchrony is 79%, G2 is 90%.

### Example 3

### Synchronisation of Arabidopsis Cell Suspension culture MM1 by Combined Treatment with Aphidicolin and Propyzamide

To reversible arrest cells in mitosis, cells were first blocked with aphidicolin, followed by propyzamide according to Nagata et al. 1992 and samples taken at various time points to determine the mitotic index and labelling index as described in Example 2A.

The aphidicolin block of the Arabidopsis cell suspension culture was done as described in Example 2A. After release of aphidicolin, the cells were resuspended in 250 ml fresh MSS-medium and incubated for about 9 hours under cultivation conditions as described in Example 2A. 1.6 μg/ml propyzamide was added to block cells in mitosis, and the cell suspension was further incubated for about 6 hours. The same washing procedure was used to remove propyzamide and the cells were incubated under cultivation conditions as described in Example 2A.

The synchrony achieved was monitored after release of propyzamide, by determining the labelling index and the mitotic index of samples taken at various time points. Blocking the cells with aphidicolin and propyzamide revealed a MI peak of 20% and a labelling index of about 50%. (Table III).

This procedure is useful for examining the cell cycle phase transitions M-G1-S.

Table III. Data obtained from analysis of samples from the experiment described in Example 3. Samples were collected at the time indicated (time of removal of propyzamide equals time point 0), and analysed for MI and LI, using the methods described in Example 2A.

| Time (h) | LI (%) MM1 | MI (%) MM1 |
|---|---|---|
| 0 | 1.71 | 20.48 |
| 1 | 2.33 | 16.89 |
| 2 | 4.33 | 12.78 |
| 3 | 5.68 | 7.7 |
| 4 | 12.57 | 1.64 |
| 5 | 15.49 | 1.45 |
| 6 | 15.75 | 1.52 |
| 7 | 21.67 | 1.58 |
| 8 | 23.47 | 1.72 |
| 9 | 24.17 | 1.37 |
| 10 | 43.85 | 2.1 |
| 11 | 50.65 | 1.48 |
| 12 | 42.38 | 2.28 |
| 13 | 36.89 | 2.02 |
| 14 | 23.85 | 2.11 |

### Example 4

### Scaled-up Procedure Suitable for Analysis of Expression Level of Cell Cycle Regulated Genes in the Arabidopsis Cell Suspension Culture MM1 (DSM 13563) Synchronised with Aphidicolin

The level of synchronicity of the Arabidopsis cell suspension culture MM1 (DSM 13563)

was analysed following the expression level of known cell cycle regulated genes. Synchronisation was performed as described in Example 2A, with the exception that two 7-day old cultures of the weekly sub-cultured cell suspension (210 ml each) were combined. 40 ml of combined culture was transferred into 200 ml of fresh MSS-medium in each of 10 separate flasks. After incubation for 24 hrs under conditions as described in Example 1, in the presence of 4.16 μg/ml aphidicolin, cells from 5 pooled flasks were washed through a nylon net (mesh size 47 μm) and washed with 2 litre MSS medium and shaken vigorously to remove the aphidicolin. Cells were transferred into a 50 ml plastic tube, washed again with MSS medium and centrifuged at 2500 rpm for 1 min (1075 rcf). Cells were resuspended and combined with the cells from the other 5 flasks similarly washed in a total volume of 300 ml fresh MSS-medium. 30 ml of this cell suspension was then added to 200 ml fresh MSS-medium in each of 10 separate flasks.

For every time point a 30 ml sample was split into:

1) 25 ml for Northern blot (transferred into 50 ml plastic tube, centrifuged at 2500 rpm for 1 min, the supernatant discarded, and the cell pellet shock-frozen in liquid nitrogen before storing in −80° C. freezer)

2) 5 ml for LI and Ml determination as described in Example 2A.

A MI peak of 9.8% was observed, about 12 hrs after release from the aphidicolin block. Labelling index was also determined and reached a peak of 52%, 3 h after release.

The quantitated values of RNA Northern expression analysis of different cell cycle regulated genes are summarised in Table IV. Samples were collected at the time point indicated (time of removal of aphidicolin equals time point 0 hrs) and were analysed for expression of the following genes by Northern blot:

histone H4 (ID/Accession no. ATH4 GB/M17133)

CycB1 (Accession no. X62279)

CDK-a (also called Cdc2a/CDK2a: Accession no. ATCDC2AP)

CDK-b1 (also called Cdc2b/CDK2b: Accession no. ATCDC2BP)

CDK-b2 (Accession no. AC007369/gene F9H16.8)

CycD2 (Accession no. X83370)

CycD3 (Accession no. X83371)

For each probe the maximum hybridisation signal obtained was defined as 100%.

TABLE IV
Quantitated values of RNA Northern expression analysis of different cell cycle regulated genes from aphidicolin
synchronisation experiment.

| probe | histone H4 | | cycB1 | | CDK-a | | CDK-b1 | | CDK-b2 | | CycD2 | | CycD3 | |
|---|---|---|---|---|---|---|---|---|---|---|---|---|---|---|
| Time | Band | | Band | | Band | | Band | | Band | | Band | | Band | |
| point | in- | | in- | | in- | | in- | | in- | | in- | | in- | |
| (hrs) | tensity | % | tensity | % | tensity | % | tensity | % | tensity | % | tensity | % | tensity | % |
| 0 | 1029 | 33.81 | 636 | 33.18 | 321 | 17.71 | 500 | 34.26 | 1084 | 7.78 | 588 | 11.06 | 465 | 12.10 |
| 2 | 3046 | 100.0 | 1201 | 62.69 | 687 | 37.87 | 844 | 57.86 | 1796 | 12.90 | 984 | 18.49 | 555 | 14.46 |
| 4 | 2580 | 84.73 | 902 | 47.09 | 818 | 45.09 | 1018 | 69.78 | 1144 | 8.22 | 773 | 14.53 | 1674 | 43.55 |
| 6 | 1794 | 58.91 | 1143 | 59.67 | 1202 | 66.22 | 1085 | 74.34 | 1683 | 12.09 | 1103 | 20.73 | 2933 | 76.31 |
| 8 | 987 | 32.42 | 1330 | 69.42 | 1054 | 58.06 | 1160 | 79.46 | 3963 | 28.47 | 1151 | 21.64 | 2798 | 72.80 |
| 10 | 1178 | 38.70 | 1607 | 83.86 | 1406 | 77.44 | 1407 | 96.41 | 6947 | 49.90 | 1704 | 32.02 | 2964 | 77.13 |
| 12 | 868 | 28.53 | 1717 | 89.57 | 1456 | 80.20 | 1450 | 99.31 | 12174 | 87.45 | 2716 | 51.04 | 3288 | 85.55 |
| 14 | 694 | 22.81 | 1917 | 100.0 | 1678 | 92.43 | 1459 | 100.0 | 13921 | 100 | 4105 | 77.14 | 3844 | 100.0 |
| 16 | 388 | 12.75 | 1466 | 76.51 | 1435 | 79.03 | 1078 | 73.89 | 7774 | 55.84 | 3539 | 66.51 | 3052 | 79.41 |
| 18 | 343 | 11.26 | 1740 | 90.81 | 1816 | 100.0 | 751 | 51.49 | 9407 | 67.57 | 5322 | 100.0 | 3040 | 79.09 |
| 20 | 591 | 19.43 | 1476 | 77.04 | 1620 | 89.25 | 859 | 58.87 | 6654 | 47.80 | 4464 | 83.89 | 3615 | 94.06 |
| 22 | 534 | 17.55 | 864 | 45.09 | 972 | 53.55 | 614 | 42.07 | 3172 | 22.78 | 2481 | 46.62 | 2283 | 59.40 |
| 24 | 541 | 17.77 | 933 | 48.70 | 621 | 34.25 | 447 | 30.65 | 1912 | 13.73 | 3429 | 64.44 | 1019 | 26.53 |
| 26 | 701 | 23.04 | 942 | 49.18 | 743 | 40.96 | 726 | 49.74 | 3183 | 22.86 | 3648 | 68.56 | 1987 | 51.71 |
| 28 | 437 | 14.35 | 1012 | 52.79 | 659 | 36.32 | 742 | 50.87 | 2583 | 18.55 | 3724 | 69.98 | 2490 | 64.80 |
| 30 | | | | | 532 | 29.30 | 839 | 57.47 | 3089 | 22.19 | 3334 | 62.66 | 2726 | 70.93 |

### Example 5

### Synchronisation of MM1 by Sucrose Deprivation of Cells 7 Days After Previous Subculture, and Analysis of RNA Samples by Northern Blots

The principle use of this procedure is to study the re-entry into the cell cycle of non-dividing cells, and their behaviour over the phases of the cell cycle G1-S-M.

The treatment of the cells before and during the sucrose deprivation step seems to be important for the degree of synchronicity obtained.

Synchronisation of MM1 early stationary phase cells (D7). Procedure:

Arabidopsis cell suspension culture MM1 was maintained as described in Example 1 under continuous light conditions (average 1300 Lux) and the cell suspension was rotated at 120 rpm at 23-25° C. To achieve synchronisation, 200 ml of an early stationary phase cell suspension (7 days after previous subculture) was washed with 11 MS-medium (MS-salt, 0.5 mg/l NM, 0.05 mg/l kinetin; lacking sucrose) by vacuum filtration (Whatman Membrane filter systems, glass holders; Cat no. 1960-004) by gently stirring with a plastic pipette to ensure that the cells were evenly washed and sucrose removed. The cells were left in a minimal volume of medium before being resuspended in approximately 200 ml MS-medium by gently stirring with a plastic pipette. 4 equal flasks (Erlenmeyer 500 ml) were set-up by transferring 40 ml of this resuspended cell suspension into 210 ml fresh MS-medium each to achieve a diluting factor of approximately 1:5 and incubated at 23° C., 120 rpm in the light for 24 hours. After 24 hours of sucrose starvation, sucrose was re-added to a final concentration of 3%. The culture was incubated under cultivation conditions as above, and at hourly intervals samples were withdrawn for RNA extraction and for LI and MI determination. LI/MI results are summarised in Table V, and quantitated values of RNA expression by Northern analysis for sample cell cycle regulated genes are presented in Table VI.

Expression of the following genes was analysed:

histone H4 (ID/Accession no. ATH4 GB/M17133)

CycB1 (Accession no. X62279)

CDK-a (also called Cdc2a/CDK2a: Accession no. ATCDC2AP)

CDK-b1 (also called Cdc2b/CDK2b: Accession no. ATCDC2BP)

CDK-b2 (Accession no. AC007369/gene F9H16.8)

CycD2 (Accession no. X83370)

CycD3 (Accession no. X83371)

At-FL39 inhibitor (FL39, described in WO 99/14331)

At-FL66 inhibitor (FL66, described in WO 99/14331)

At-ICK1 inhibitor (Accession no. ATU94772)

TABLE V
Samples were taken at the indicated time points after adding sucrose
at time 0 hrs and LI and MI were determined.

| Time point | | |
|---|---|---|
| (hours) | LI (%) | MI (%) |
| 0 | 0.6 | 0.2 |
| 1 | 0.4 | 0.2 |
| 2 | 0.8 | 0.2 |
| 3 | 1.2 | 0.4 |
| 4 | 1.2 | 0.6 |
| 5 | 2.8 | 0.4 |
| 6 | 3.6 | 0.4 |
| 7 | 4.3 | 0.6 |
| 8 | 4.8 | 0.6 |
| 9 | 10.26 | 0.8 |
| 10 | 9.38 | 1.78 |
| 11 | 10.71 | 2.25 |
| 12 | 15.29 | 4.45 |
| 13 | 16.04 | 5.28 |
| 14 | 21.66 | 6.35 |
| 15 | 22.75 | 7.15 |
| 16 | 15.64 | 11.27 |
| 16.5 | — | 12.90 |
| 17 | 16.12 | 13.62 |
| 17.5 | — | 15.96 |
| 18 | 13.87 | 11.65 |
| 18.5 | — | 10.43 |
| 19 | 12.70 | 9.95 |
| 19.5 | — | 6.41 |
| 20 | 13.39 | 6.11 |
| 21 | 12.64 | 5.95 |
| 22 | 13.21 | 4.98 |
| 23 | 12.60 | 6.23 |
| 24 | 10.48 | 5.35 |
| 25 | 5.75 | 6.13 |

Table VI shows data from analysis of samples obtained in the experiment described in Example 5. Samples were
collected at the time indicated (time of addition of sucrose = 0 hrs) and were analysed for expression
of the genes indicated by Northern blot. For each probe, the maximum hybridisation signal obtained
was defined as 100%.

| Gene | | | | | | | | | | |
|---|---|---|---|---|---|---|---|---|---|---|
| probe | | | | | | | | | | |
| Time | histone H4 | | cycB1 | | CDK-a | | CDK-b1 | | CDK-b2 | |
| point | Band | | Band | | Band | | Band | | Band | |
| (hours) | intensity | percentage | intensity | percentage | intensity | percentage | intensity | percentage | intensity | percentage |
| 0 | 187.10 | 6.40 | 241.54 | 1.71 | 441.76 | 23.51 | 163.71 | 9.66 | 106.81 | 9.16 |
| 2 | 223.36 | 7.65 | 799.28 | 5.64 | 941.06 | 50.08 | 162.27 | 9.57 | 217.49 | 18.65 |
| 4 | 186.66 | 6.39 | 168.88 | 1.19 | 969.83 | 51.61 | 174.07 | 10.26 | 81.46 | 6.99 |
| 6 | 420.25 | 14.39 | 243.54 | 1.72 | 1079.10 | 57.43 | 291.13 | 17.17 | 78.59 | 6.74 |
| 8 | 776.21 | 26.57 | 250.56 | 1.77 | 1110.86 | 59.12 | 338.35 | 19.96 | 69.37 | 5.94 |
| 10 | 2337.99 | 80.04 | 732.13 | 5.17 | 1131.59 | 60.22 | 1199.64 | 70.77 | 101.61 | 8.71 |
| 12 | 2271.91 | 77.78 | 1525.89 | 10.78 | 1879.32 | 100 | 1695.66 | 100 | 162.15 | 13.91 |
| 14 | 2827.43 | 96.80 | 4447.89 | 31.43 | 1379.28 | 73.40 | 1219.95 | 71.97 | 381.64 | 32.73 |
| 16 | 2921.54 | 100.00 | 5307.30 | 37.51 | 1421.86 | 75.67 | 1236.94 | 72.97 | 329.50 | 28.26 |
| 18 | 2569.58 | 87.97 | 11298.11 | 79.84 | 1139.81 | 60.66 | 938.32 | 55.36 | 757.29 | 64.95 |
| 20 | 2466.35 | 84.43 | 11738.59 | 82.94 | 1332.20 | 70.90 | 711.26 | 41.96 | 809.09 | 69.39 |
| 22 | 2226.75 | 76.23 | 14150.95 | 100 | 1331.27 | 70.85 | 596.94 | 35.22 | 1166.27 | 100 |
| Gene | | | | | | | | | | |
| probe | | | | | | | | | | |
| Time | CycD2 | | cycD3 | | At-FL39 inhibitor | | At-FL66 inhibitor | | At-ICK1 inhibitor | |
| point | Band | | Band | | Band | | Band | | Band | |
| (hours | intensity | percentage | intensity | percentage | intensity | percentage | intensity | percentage | intensity | Percentage |
| 0 | 147.48 | 8.33 | 310.05 | 4.92 | 1966.46 | 28.51 | 0 | 0 | 1682.23 | 97.46 |
| 2 | 237.10 | 13.39 | 308.02 | 4.89 | 6898.81 | 100 | 442.63 | 13.04 | 770.91 | 44.66 |
| 4 | 355.23 | 20.07 | 399.91 | 6.35 | 1540.95 | 22.34 | 563.76 | 16.61 | 520.40 | 30.15 |
| 6 | 552.43 | 31.21 | 2830.18 | 44.92 | 546.77 | 7.93 | 1359.51 | 40.04 | 913.47 | 52.92 |
| 8 | 747.05 | 42.21 | 3541.49 | 56.20 | 163.80 | 2.37 | 1461.02 | 43.03 | 803.28 | 46.54 |
| 10 | 755.94 | 42.71 | 4353.90 | 69.10 | 0 | 0 | 1696.81 | 49.98 | 919.24 | 53.26 |
| 12 | 1770.52 | 100.00 | 6301.57 | 100.00 | 0 | 0 | 3395.91 | 100 | 1726.99 | 100 |
| 14 | 1615.64 | 91.28 | 5396.82 | 85.65 | 0 | 0 | 3091.55 | 91.06 | 1666.56 | 96.56 |
| 16 | 1500.08 | 84.75 | 5524.21 | 87.67 | 0 | 0 | 2748.91 | 80.97 | 1092.70 | 63.31 |
| 18 | 957.05 | 54.07 | 5242.21 | 83.20 | 0 | 0 | 3183.73 | 93.78 | 724.34 | 41.97 |
| 20 | 1044.59 | 59.02 | 5404.02 | 85.76 | 0 | 0 | 2996.32 | 88.26 | 417.34 | 24.18 |
| 22 | 1059.05 | 59.83 | 4317.95 | 68.53 | 0 | 0 | 1902.32 | 56.03 | 584.27 | 33.85 |

### Example 6

### Synchronisation of MM2d by Sucrose Deprivation of Cells 4 Days After Previous Sub-Culture

Arabidopsis cell suspension culture MM2d (DSM 13564) was used for this synchronisation experiment. The suspension culture was maintained by sub-culturing weekly 5 ml into 100 ml fresh MSS-medium (MS-salt, 3% sucrose, 0.5 mg/l NAA, 0.05 mg/l kinetin). The cell suspension culture was grown under continuous dark conditions and is rotated at 130 rpm, at 27° C. To achieve synchronisation, mid-exponential phase cells were starved of sucrose, which was subsequently re-added. The level of synchronicity of the Arabidopsis cell suspension culture MM2d (DSM 13564) was analysed by determining the LI/MI-index as well by following cell cycle transition using flow cytometry.

In total, 600 ml exponential phase cell suspension (4 days after previous subculture), consisting of 6 individual flasks with 100 ml culture in each, were centrifuged (688 g/2 min/without brake) in sterile 50 ml plastic tubes. The cell pellets were washed twice with 50 ml MS-medium (MS-salt, 0.5 mg/l NAA, 0.05 mg/l kinetin, lacking sucrose) by gently resuspending and repeating the centrifugation step. Cells were finally resuspended and pooled in a total volume of 600 ml MS-medium. 26 equal flasks (Erlenmeyer, 300 ml) were set-up by transferring 20 ml of the pooled resuspended cell suspension into 100 ml fresh MS-medium each to achieve a diluting effect of approximately 1:5 and incubated at 27° C., 130 rpm in the dark.

After 24 hours sucrose starvation, sucrose was re-added to a final concentration of 3%, incubated under cultivation conditions as above and samples taken at various time points to determine the mitotic index (DAPI) and labelling index (BrdU). Cell cycle progression was monitored by flow cytometry and determination of LI and MI.

TABLE VII
Data from analysis of samples obtained in the experiment described in
Example 6. Samples were collected at the time indicated (time of
sucrose addition = 0 hrs), and analysed for the proportion of G1, S
and G2 cells by flow cytometry, and for MI and LI as described in
Example 2A. The proportion of G1, S and G2 cells in mid-exponential
(D4) cells is indicated for comparison.

| | G1 (%) | S (%) | G2 (%) | LI (%) | MI (%) |
|---|---|---|---|---|---|
| Time (h) | MM2d | MM2d | MM2d | MM2d | MM2d |
| D4 | 53.9 | 5.9 | 40.2 | | |
| 0 | 66.9 | 2.9 | 30.3 | 0 | 0 |
| 1 | 66 | 7 | 26.9 | 0.2 | 0.2 |
| 2 | 64.8 | 7.3 | 27.9 | 0.8 | 0.2 |
| 3 | 62.8 | 4.6 | 32.7 | 2.9 | 0.2 |
| 4 | 59.9 | 9.8 | 30.3 | 4.22 | 0.2 |
| 5 | 54.4 | 13.9 | 31.7 | 7.91 | 0.6 |
| 6 | 49.9 | 14.3 | 35.8 | 11.16 | 0.6 |
| 7 | 44.3 | 15.5 | 40.2 | 13.26 | 0.8 |
| 8 | 41.5 | 18.6 | 39.9 | 17.42 | 0.71 |
| 9 | 35.6 | 19.8 | 44.6 | 21 | 0.75 |
| 10 | 36.2 | 17.3 | 46.5 | 22.12 | 1.77 |
| 11 | 35.6 | 14.4 | 50 | 17.72 | 2.39 |
| 12 | 34.6 | 9.3 | 56 | 14.73 | 3.57 |
| 13 | 39 | 7.9 | 53.1 | 12.05 | 5.13 |
| 14 | 42 | 5.9 | 52.1 | 7.86 | 5.54 |
| 15 | 47.9 | 5.1 | 47 | 8.23 | 7.76 |
| 16 | 55 | 3.4 | 41.6 | 3.08 | 8.9 |
| 17 | 55.4 | 4.2 | 40.4 | 3.12 | 9.96 |
| 18 | 56.6 | 3.3 | 40.1 | 2.14 | 10.7 |
| 19 | 57.4 | 4.9 | 37.7 | 2.03 | 8.51 |
| 20 | 61.5 | 6.4 | 32.1 | 6.09 | 6.09 |
| 21 | 60.5 | 5.1 | 34.4 | 7.54 | 4.52 |
| 22 | 63 | 6 | 30.9 | 7.17 | 2.86 |
| 23 | 54.9 | 11.1 | 34 | 9.74 | 2.61 |
| 24 | 52.9 | 7.6 | 39.5 | 6.82 | 2.17 |
| 25 | 54.5 | 14.4 | 31 | 6.71 | 2.13 |

### Example 7

### Transformation of MM1 with At-cycD2-overexpressing Construct

Arabidopsis cell suspension culture MM1 (DSM 13563) was used for transformation experiments. The suspension was maintained by sub-culturing weekly 7.5 ml into 200 ml of fresh MSS-medium (MS-salt, 3% sucrose, 0.5 mg/l NM, 0.05 mg/l kinetin) which is cultured in 500 ml narrow neck Erlenmeyer flasks, of which the opening is covered with double layer of aluminium foil and loosely covered with Saran wrap and which are placed on a GYROTORY® Shaker (New Brunswick Scientific). The cell suspension culture was grown under continuous light conditions (24 hrs a day); Philips lamps were used with the specification TLD50W/84oHF; light level is in average 1300 Lux and the cell suspension culture is rotated at 120 rpm at a temperature varying between 23-25° C.

An overexpression construct comprising the cycD2 coding region of Arabidopsis thaliana under the control of CaMV35S promoter, was introduced into cell line MM1 by Agrobacterium-mediated transformation as follows:

2.5 ml of an early stationary phase cell suspension MM1 (7 days after previous subculture) was subcultured into 25 ml MSS-medium (dilution 1:10) and incubated at 23° C., 120 rpm in the conditions as described above for MM1, except that the suspension was cultured in 100 ml narrow neck Erlenmeyer flasks, for a period of 2 days. An Agrobacterium culture (LB4404 containing a T-DNA vector with a chimeric gene comprising a CaMV35S promoter and an operably linked DNA region encoding cyclin D2 from Araoidopsis thaliana was inoculated in 50 ml LB-broth (containing 100 μg/ml spectinomycin) and incubated at 30° C., 130 rpm. The optical density (OD) was determined by measuring the absorbance at 600 nm with an UV-spectrophotometer (BIORAD, SmartSpec™3000). 100 μl of exponential growing Agrobacterium culture (OD600 nm between 1 and 1.5) was washed three times with 1 ml MSS-medium by centrifugation (13000 rpm/2 min) and resuspended in 900 μl MSS-medium (dilution 1:10). 10 ml of the exponential growing MM1 cell suspension (2 days old) was subcultured into 50 ml fresh MSS-medium (dilution 1:5). This culture was used in part for co-cultivation with Agrobacterium and in part for production of conditioned medium. For co-cultivation, 10 ml of diluted (1:5) MM1 suspension was transferred into a 250 ml Erlenmeyer flask, acetosyringone (3′,5′-dimethoxy-4′-hydroxyacetophenone, Acros Organics) was added to a concentration of 500 μM and the cell suspension was infected with 100 μl of a diluted (1:10) Agrobacterium culture. The Agrobacterium-infected MM1 culture was incubated under continuous light conditions (1300 Lux), at 23° C. without agitation for 53 hours. To obtain conditioned medium, the remaining diluted cell suspension was incubated at 120 rpm, 23° C. under continuous light for further 2 days. Cells were cleared from conditioned medium by centrifugation (1500 rpm/5 min/without brake; 387 rcf). The cell culture mixed with the Agrobacterium cells was transferred into a 50 ml falcon tube and centrifuged for 5 min at 1500 rpm, without applying brake force. The cells were gently washed three times with 50 ml fresh MSS-medium (1500 rpm/5 min/without brake; 387 rcf) and finally resuspended in 20 ml conditioned medium. Timentin (Tricarcillin/Clavulanic Acid, 1500/100 mg; Duchefa Biochemie BV) was added to a concentration of 500 μg/ml to select against remaining Agrobacteria and the putative transformed MM1 cells were incubated for further 3 days rotating at 120 rpm, 23° C. under continuous light conditions (1300 Lux) in a 250 ml Erlenmeyer flask. The cell suspension was transferred into a 50 ml Falcon tube, centrifuged for 5 min at 1500 rpm, without applying brake force and resuspended in 10 ml fresh MSS-medium (supplemented with 50 μg NM and 5 μg kinetin). 2.5 ml of cell suspension was spread each of three plates containing 0.8% MSS-agar (containing no NM or kinetin), 250 μg/ml Timentin and 100 μg/ml kanamycin and 2.5 ml of cell suspension on MSS-agar (without antibiotics, as control). Plates were sealed with tape (3M, Micropore™) and incubated under continuous light conditions (1300 Lux), at 23° C. After 2-3 weeks putative transformed calli were transferred onto new MSS-agar plates (250 μg/ml Timentin, 100 μg/ml kanamycin) and left to grow for further 1-2 weeks to get enough material for inoculation.

To re-establish liquid cultures of putative transgenic MM1-calli, callus tissue was transferred to a 55 mm petridish, carefully squeezed with a pipet tip and resuspended in 10 ml fresh MSS-medium (0.5 mg/l NM, 0.05 mg/l kinetin, 100 μg/ml kanamycin). The callus derived cell suspensions were incubated at 120 rpm, 23° C. under continuous light conditions in 100 ml Erlenmeyer flasks. After 10 days cultivation, 10 ml MSS-medium (0.5 mg/l NM, 0.05 mg/l kinetin) was added to the suspension cultures and left for further 7 days under conditions as above. 5 ml putative transgenic cell suspension was subcultured into 50 ml fresh MSS-medium (0.5 mg/l NM, 0.05 mg/l kinetin, 100 μg/ml kanamycin). This subculturing procedure was repeated twice at 7 days intervals in the same medium. Subsequent subculturing was carried out weekly into fresh MSS-medium (0.5 mg/l NAA, 0.05 mg/l kinetin without kanamycin selection), adjusting the transferred volume of cells to produce an inoculum of approximately the same as WT-MM1 culture. In the example of putative transgenic MM1-cycD2 cell suspensions, these were maintained by subculturing weekly 3.5 ml into 100 ml fresh MSS-medium (0.5 mg/l NAA, 0.05 mg/l kinetin) in 300 ml Erlenmeyer flasks. The same procedure can be followed with MM2d except cultures are incubated under dark conditions. Putative transgenic calli or the suspension cultures thereof were analyzed using PCR and appropriate primers allowing to detect the cycD2 coding region. The amplification product of the endogenous cycD2 gene has a size of about 700 nt, whereas the amplification product of the chimeric cycD2 gene (which does not comprise the introns) has a size of about 400 bp. Transgenic MM1-cycD2 cell lines were submitted to a synchronization regime using aphidicolin block/release and could be synchronized in a similar way as non transgenic MM1 cell lines. A higher level of cycD2 RNA was found in the transgenic cell lines compared to the non transgenic cells by Northern analysis after sucrose starvation and release. Western blot analysis of sucrose starved synchronised cells revealed higher cycD2 protein level after sucrose addition than for non-transgenic cell lines.

### REFERENCES

Bartels et al. (1997) (eds.) The Yeast two hybrid system. Oxford University Press.

Binarova et al. (1998) The Plant Journal, 16(6): 697-707

Callard et al. (1997) Plant Physiology, 115: 1385-1395

Fuerst et al. (1996) Plant Physiology, 112: 1023-1033

Fukuda et al. (1994) International Journal of Developmental Biology, 38: 287-299

Galbraith DW. et al. (1991) Plant Physiology 96: 985-989

Glab N. et al. (1994) FEBS Letters 353: 207-211

Gratzner HG et al. (1975) Experimental Cell Research 95: 88-94

Ito M et al.(1997) The Plant Journal, 11 (5): 983-992

Juan G. and Darzynkiewicz Z. (1998) Cell cycle analysis by flow and laser scanning cytometry in Cell Biology: a laboratory handbook, second edition, vol 1, Academic Press

Kalejta et al. (1997). Experimental Cell Research, 231: 173-183

Komari (1989) Plant Science 60: 223-229

Kulp et al. (1996) Experimental Cell Research, 229: 60-68

Lee et al. (1988) PNAS, 88:6389

Levi M. et al. (1987) Physiologia Plantarum 71: 68-72

Mader J C. and Hanke D E. (1996) Plant Growth Regulation 15:95-102

Magyar et al. (1997) The Plant Cell, 9: 223-235

May et al. (1993) Plant Physiology, 103: 621-627

Meijer et al. (1996) Trends in Cellbiology, 6: 393-397

Miyake T et al. (1997) Journal of Plant Physiology 150:528-536

Nagata et al. (1992) International Review of Cytology, 132: 1-30

Perennes et al. (1993) FEBS Letters, 333 (1-2): 141-145

Planchais et al. (1997) The Plant Journal, 12(1): 191-202

Reichfeld et al. (1999) The Plant Journal, 17(6): 647-656

Riou-Khamlichi et al. (1999) Science, 283: 1541-1544

Samuels et al.(1998) Protoplasma, 202: 232-236

Songstad et al. (1995) Plant Cell, Tissue and Organ Culture 40: 1-15

Trethewey et al. (1999) Curr. Opin. Plant Sci. 2: 83-85

Vemoux T. et al. (2000) The Plant Cell 12: 97-109

1. A method for producing a synchronised Arabidopsis cell suspension culture comprising the steps of:
a.) arresting a suitable number of cells of an Arabidopsis cell suspension culture in a distinct stage of the cell cycle by a reversible block;
b.) removing said reversible block to allow said arrested cells to progress through said cell cycle; and
c.) further culturing said Arabidopsis cell suspension culture under suitable conditions;
wherein said Arabidopsis cell suspension culture has a cell doubling time of less than about 30 hrs.

2. The method of claim 1, wherein said Arabidopsis cell suspension culture is capable of producing chlorophyll under suitable light conditions.

3. The method of claim 1 or 2, wherein said Arabidopsis cell suspension culture is culture MM1 (DSM 13563) or MM2d (DSM 13564), or a derivative thereof.

4. The method of any one of claim 1 to 3, wherein said Arabidopsis cell suspension culture is an early stationary phase cell suspension or a mid-exponential phase cell suspension, which has been diluted, preferably five to seven fold diluted, prior to said arresting by said reversible block.

5. A method for producing a synchronised Arabidopsis cell suspension culture comprising the steps of:
a.) contacting an Arabidopsis cell suspension culture with a first cell cycle blocking compound, in a concentration and for a period of time sufficient to arrest the cells of said cell suspension culture in a distinct stage of the cell cycle;
b.) removing said cell cycle blocking compound from said cell suspension culture after said period of time to produce a synchronised culture; and
c.) incubating said synchronised culture under suitable culture conditions; wherein said Arabidopsis cell suspension culture has a cell doubling time of less than 30 hrs.

6. The method of claim 5, wherein said Arabidopsis suspension culture is the Arabidopsis cell suspension culture MM1 or MM2d, respectively deposited under DSM number 13563 or DSM number 13564 or a derivative thereof.

7. The method of claim 5 or 6, wherein said first cell cycle blocking compound is selected from aphidicolin, propyzamide, roscovitine, olomoucine, mimosine, quercitine, abscisic acid, or cycloheximide.

8. The method of claim 7 wherein said first cell cycle blocking compound is roscovitine.

9. The method of claim 7, wherein said first cell cycle blocking compound is aphidicolin.

10. The method of any one of claims 5 to 9, further comprising the steps of
a.) contacting said synchronised cell suspension culture with a second cell cycle blocking compound in a concentration and for a period of time sufficient to arrest a suitable number of cells of said cell suspension culture in a distinct stage of the cell cycle;
b.) removing said second cell cycle blocking compound from said cell suspension culture after said period of time to yield a synchronised culture; and
c.) incubating said synchronised culture under suitable culture conditions;
wherein said second cell cycle blocking compound is capable of arresting cells of said cell suspension culture in a another stage of the cell cycle than said stage wherein said cells are blocked by contacting with said first cell cycle blocking compound.

11. The method of claim 10, wherein said second cell cycle blocking compound is selected from aphidicolin, propyzamide, roscovitine, olomoucine, mimosine, quercitine, or abscisic acid.

12. The method of claim 10 wherein said first cell cycle blocking compound is aphidicolin and said second cell cycle blocking compound is propyzamide.

13. The method of any one of claims 5 to 12, wherein said concentration of said first cell cycle blocking compound is about 0.2 μg/ml to about 170 μg/ml.

14. The method of any one of claims 5 to 12, wherein said concentration of said first cell cycle blocking compound is about 2 to about 20 μg/ml.

15. The method of any one of claims 10 to 12, wherein said concentration of said second cell cycle blocking compound is about 0.2 μg/ml to about 170 μg/ml.

16. The method of any one of claims 10 to 12, wherein said concentration of said second cell cycle blocking compound is about 2 to about 20 μg/ml.

17. A method for producing a synchronised Arabidopsis cell suspension culture comprising the steps of:
a.) incubating an Arabidopsis cell suspension culture in a culture medium lacking one or more of the culture medium compounds required for the growth of said cell suspension culture for a period of time sufficient to arrest a suitable number of cells of said cell suspension culture in a distinct stage of the cell cycle;
b.) replacing said culture medium lacking said culture medium compound required for the growth of the suspension culture with a medium comprising said required compound or a compound with similar function; and
c.) incubate the cell suspension culture under suitable culture conditions to produce a synchronised cell suspension culture;
wherein said Arabidopsis cell suspension culture has a cell doubling time of less than about 30 hrs.

18. The method of claim 17, wherein said Arabidopsis cell suspension culture is capable of producing chlorophyll under suitable light conditions.

19. The method of claim 17, wherein said Arabidopsis cell suspension culture is culture MM1 (DSM 13563) or MM2d (DSM 13564), or a derivative thereof.

20. The method of claim 17, wherein said Arabidopsis cell suspension culture is an exponentially growing or early stationary phase cell suspension, which is diluted, preferably five to seven fold diluted, prior to said arresting by said reversible block.

21. The method of any one of claims 17 to 20, wherein said compound required for the growth of the suspension culture is selected from a carbohydrate, a nitrate, a phosphate, an auxin or a cytokinin.

22. The method of claim 21, wherein said compound required for the growth of the suspension culture is selected from sucrose, potassium nitrate, potassium dihydrogen phosphate, nicotine acetamide or kinetin.

23. The method of any one of claims 17 to 22, further comprising the steps of
a.) contacting said synchronised cell suspension culture with a cell cycle blocking compound in a concentration and for a period of time sufficient to arrest a suitable number of cells of said cell suspension culture in a distinct stage of the cell cycle;
b.) removing said cell cycle blocking compound from said cell suspension culture after said period of time to yield a synchronised culture; and
c.) incubating said synchronised culture under suitable culture conditions;

24. Arabidopsis cell suspension culture MM1 (DSM 13563).

25. Arabidopsis cell suspension culture MM2d (DSM 13564).

26. A synchronised Arabidopsis cell suspension culture wherein said cell suspension culture has a maximum labelling index of at least 15% or a maximum mitotic index of at least 9% when cells of said suspension culture are progressing through the cell cycle.

27. A synchronised Arabidopsis cell suspension culture which is obtainable by the methods of any of the claims 1 to 23, wherein said cell suspension culture has a maximum labelling index of at least 15% or a maximum mitotic index of at least 9% when cells of said suspension culture are progressing through the cell cycle.

28. A synchronised Arabidopsis cell suspension culture wherein synchrony is maintained for at least one additional cell cycle phase after the completion of the cell cycle phase in which the cells of the cells suspension culture are blocked.

29. The synchronised Arabidopsis cell suspension culture of claim 28, wherein synchrony is maintained for at least two additional cell cycle phases after the completion of the cell cycle phase in which the cells of the cells suspension culture are blocked.

30. A method for isolating a cell cycle regulated gene comprising the steps of
a.) producing a first sample of a synchronised Arabidopsis cell suspension culture wherein said cell suspension culture has a maximum labelling index of at least 15% or a maximum mitotic index of at least 9% when cells of said suspension culture are progressing through the cell cycle;
b.) producing a second sample of a synchronised Arabidopsis cell suspension culture wherein said cell suspension culture has a maximum labelling index of at least 15% or a maximum mitotic index of at least 9% when cells of said suspension culture are progressing through the cell cycle;
c.) extracting RNA from said first and said second sample;
d.) identifying an RNA molecule with a different concentration in cells of said first sample when compared to cells of said second sample; and
e.) isolating a gene corresponding to said identified RNA molecule.

31. The method of claim 30, wherein said synchronised Arabidopsis cell suspension cultures have been obtained according to the methods of any one of claims 1 to 23.

32. The method of claim 30, wherein said RNA is polyadenylated RNA.

33. The method of claim 30 or 31, wherein said synchronised Arabidopsis cell suspension culture is produced by the method of claim 9.

34. The method of claim 30 or 31, wherein said synchronised Arabidopsis cell suspension culture is produced by the method of claim 12.

35. A method for isolating a cDNA corresponding to a cell cycle regulated gene comprising the steps of
a.) producing a first sample of a synchronised Arabidopsis cell suspension culture wherein said cell suspension culture has a maximum labelling index of at least 15% or a maximum mitotic index of at least 9% when cells of said suspension culture are progressing through the cell cycle;
b.) producing a second sample of a synchronised Arabidopsis cell suspension culture wherein said cell suspension culture has a maximum labelling index of at least 15% or a maximum mitotic index of at least 9% when cells of said suspension culture are progressing through the cell cycle;
c.) extracting RNA from said first and said second sample;
d.) identifying a RNA molecule with a different concentration in cells of said first sample when compared to cells of said second sample; and
e.) isolating a cDNA corresponding to said identified RNA molecule.

36. The method of claim 35, further comprising the step of generating a transgenic plant comprising said isolated cDNA.

37. The method of any one of claim 30 to 34, further comprising the step of generating a transgenic plant comprising said isolated cell cycle regulated gene.

38. A method for analysing the timing of expression, preferably transcription, of a gene of interest during the cell cycle of an Arabidopsis cell comprising the steps of:
a.) producing at a particular time point after the release from the cell cycle block a sample of a synchronised Arabidopsis cell suspension culture wherein said cell suspension culture has a maximum labelling index of at least 15% or a maximum mitotic index of at least 9% when cells of said suspension culture are progressing through the cell cycle;
b.) extracting RNA, preferably polyadenylated RNA from the sample; and
c.) identifying the relative level of an RNA molecule hybridising with a nucleotide sequence of at least 20 consecutive nucleotides of said gene of interest.

39. A method for identifying a protein whose abundance, state of modification or enzymatic activity varies during the cell cycle comprising the steps of:
a.) producing at a particular time point after the release from the cell cycle block a sample of a synchronised Arabidopsis cell suspension culture wherein said cell suspension culture has a maximum labelling index of at least 15% or a maximum mitotic index of at least 9% when cells of said suspension culture are progressing through the cell cycle, preferably obtained through the methods of any one of claims 1 to 23;
b.) producing proteins from the sample of cells;
c.) identifying the relative level, state of modification or enzymatic activity of proteins in cells of the sample; and
d.) optionally, producing samples at other time points and reiterating steps a to c.

40. A method for analysing the relative level of a particular metabolite during the cell cycle of an Arabidopsis cell comprising the steps of
a.) producing at a particular time point after the release from the cell cycle block a sample of a synchronised Arabidopsis cell suspension culture wherein said cell suspension culture has a maximum labelling index of at least 15% or a maximum mitotic index of at least 9% when cells of said suspension culture are progressing through the cell cycle; and
b.) analysing the relative level of a particular metabolite in cells of the sample.

41. (new) A method for producing a synchronised Arabidopsis cell suspension culture comprising the steps of:
a.) arresting a suitable number of cells of an Arabidopsis cell suspension culture in a distinct stage of the cell cycle by a reversible block;
b.) removing said reversible block to allow said arrested cells to progress through said cell cycle; and
c.) further culturing said Arabidopsis cell suspension culture under suitable conditions;
wherein said Arabidopsis cell suspension culture is culture MM1 (DSM 13563) or MM2d (DSM 13564), or a derivative thereof.

42. (new) The method of claim 41, wherein said Arabidopsis cell suspension culture is an early stationary phase cell suspension or a mid-exponential phase cell suspension, which has been diluted five to seven fold prior to said arresting by said reversible block.

43. (new) A method for producing a synchronised Arabidopsis cell suspension culture comprising the steps of:
a.) contacting an Arabidopsis cell suspension culture with a first cell cycle blocking compound, in a concentration and for a period of time sufficient to arrest the cells of said cell suspension culture in a distinct stage of the cell cycle;
b.) removing said cell cycle blocking compound from said cell suspension culture after said period of time to produce a synchronised culture; and
c.) incubating said synchronised culture under suitable culture conditions;
wherein said Arabidopsis cell suspension culture is the Arabidopsis cell suspension culture MM1 or MM2d, respectively deposited under DSM number 13563 or DSM number 13564 or a derivative thereof.

44. (new) The method of claim 43, wherein said first cell cycle blocking compound is aphidicolin, propyzamide, roscovitine, olornoucine, mimosine, quercitine, abscisic acid or cycloheximide.

45. (new) The method of claim 44, wherein said first cell cycle blocking compound is roscovitine.

46. (new) The method of claim 44, wherein said first cell cycle blocking compound is aphidicolin.

47. (new) The method of claim 43, further comprising the steps of:
a.) contacting said synchronised cell suspension culture with a second cell cycle blocking compound in a concentration and for a period of time sufficient to arrest a suitable number of cells of said cell suspension culture in a distinct stage of the cell cycle;
b.) removing said second cell cycle blocking compound from said cell suspension culture after said period of time to yield a synchronised culture; and
c.) incubating said synchronised culture under suitable culture conditions;
wherein said second cell cycle blocking compound is capable of arresting cells of said cell suspension culture in a another stage of the cell cycle than said stage wherein said cells are blocked by contacting with said first cell cycle blocking compound.

48. (new) The method of claim 47, wherein said second cell cycle blocking compound is aphidicolin, propyzamide, roscovitine, olomoucine, mimosine, quercitine or abscisic acid.

49. (new) The method of claim 47, wherein said first cell cycle blocking compound is aphidicolin and said second cell cycle blocking compound is propyzamide.

50. (new) The method of claim 43, wherein said concentration of said first cell cycle blocking compound is about 0.2 μg/ml to about 170 μg/ml.

51. (new) The method of claim 43, wherein said concentration of said first cell cycle blocking compound is about 2 to about 20 μg/ml.

52. (new) The method of claim 43, wherein said concentration of said second cell cycle blocking compound is about 0.2 μg/ml to about 170 μg/ml.

53. (new) The method of claim 47, wherein said concentration of said second cell cycle blocking compound is about 2 to about 20 μg/ml.

54. (new) A method for producing a synchronised Arabidopsis cell suspension culture comprising the steps of:
a.) incubating an Arabidopsis cell suspension culture in a culture medium lacking one or more of the culture medium compounds required for the growth of said cell suspension culture for a period of time sufficient to arrest a suitable number of cells of said cell suspension culture in a distinct stage of the cell cycle;
b.) replacing said culture medium lacking said culture medium compound required for the growth of the suspension culture with a medium comprising said required compound or a compound with similar function; and
c.) incubating the cell suspension culture under suitable culture conditions to produce a synchronised cell suspension culture;
wherein said Arabidopsis cell suspension culture is culture MM1 (DSM 13563) or MM2d (DSM 13564), or a derivative thereof.

55. (new) The method of claim 54, wherein said Arabidopsis cell suspension culture is an exponentially growing or early stationary phase cell suspension, which is diluted five to seven fold prior to said arresting by said reversible block.

56. (new) The method of claim 54, wherein said compound required for the growth of the suspension culture is a carbohydrate, a nitrate, a phosphate, an auxin or a cytokinin.

57. (new) The method of claim 56, wherein said compound required for the growth of the suspension culture is sucrose, potassium nitrate, potassium dihydrogen phosphate, nicotine acetamide or kinetin.

58. (new) The method of claim 54, further comprising the steps of:
a.) contacting said synchronised cell suspension culture with a cell cycle blocking compound in a concentration and for a period of time sufficient to arrest a suitable number of cells of said cell suspension culture in a distinct stage of the cell cycle;
b.) removing said cell cycle blocking compound from said cell suspension culture after said period of time to yield a synchronised culture; and
c.) incubating said synchronised culture under suitable culture conditions;

59. (new) Arabidopsis cell suspension culture MM1 (DSM 13563) or a derivative thereof.

60. (new) Arabidopsis cell suspension culture MM2d (DSM 13564) or a derivative thereof.

61. (new) A method for isolating a cell cycle regulated gene comprising the steps of:
a.) producing a first sample of a synchronised Arabidopsis cell suspension culture, wherein said cell suspension culture has a maximum labelling index of at least 15% or a maximum mitotic index of at least 9% when cells of said suspension culture are progressing through the cell cycle;
b.) producing a second sample of a synchronised Arabidopsis cell suspension culture, wherein said cell suspension culture has a maximum labelling index of at least 15% or a maximum mitotic index of at least 9% when cells of said suspension culture are progressing through the cell cycle;
c.) extracting RNA from said first and said second sample;
d.) identifying an RNA molecule with a different concentration in cells of said first sample when compared to cells of said second sample; and
e.) isolating a gene corresponding to said identified RNA molecule, wherein said synchronised Arabidopsis cell suspension cultures are obtainable according to the method of claim 41.

62. (new) The method of claim 61, wherein said RNA is polyadenylated RNA.

63. (new) A method for isolating a cDNA corresponding to a cell cycle regulated gene comprising the steps of:
a.) producing a first sample of a synchronised Arabidopsis cell suspension culture, wherein said cell suspension culture has a maximum labelling index of at least 15% or a maximum mitotic index of at least 9% when cells of said suspension culture are progressing through the cell cycle;
b.) producing a second sample of a synchronised Arabidopsis cell suspension culture, wherein said cell suspension culture has a maximum labelling index of at least 15% or a maximum mitotic index of at least 9% when cells of said suspension culture are progressing through the cell cycle;
c.) extracting RNA from said first and said second sample;
d.) identifying a RNA molecule with a different concentration in cells of said first sample when compared to cells of said second sample; and
e.) isolating a cDNA corresponding to said identified RNA molecule;
wherein said synchronised Arabidopsis cell suspension cultures are obtainable according to the method of claim 41.

64. (new) The method of claim 63, further comprising the step of generating a transgenic plant comprising said isolated cDNA.

65. (new) A method for analyzing the timing of expression or transcription of a gene of interest during the cell cycle of an Arabidopsis cell comprising the steps of:
a.) producing at a particular time point after the release from the cell cycle block a sample of a synchronised Arabidopsis cell suspension culture, obtainable through the method of claim 41, wherein said cell suspension culture has a maximum labelling index of at least 15% or a maximum mitotic index of at least 9% when cells of said suspension culture are progressing through the cell cycle;
b.) extracting RNA or polyadenylated RNA from the sample; and
c.) identifying the relative level of an RNA molecule hybridizing with a nucleotide sequence of at least 20 consecutive nucleotides of said gene of interest.

66. (new) A method for identifying a protein whose abundance, state of modification or enzymatic activity varies during the cell cycle comprising the steps of:
a.) producing at a particular time point after the release from the cell cycle block a sample of a synchronised Arabidopsis cell suspension culture, wherein said cell suspension culture has a maximum labelling index of at least 15% or a maximum mitotic index of at least 9% when cells of said suspension culture are progressing through the cell cycle and which is obtainable through the method of claim 41;
b.) producing proteins from the sample of cells;
c.) identifying the relative level, state of modification or enzymatic activity of proteins in cells of the sample; and
d.) producing samples at other time points and reiterating steps a to c.

67. (new) A method for analyzing the relative level of a particular metabolite during the cell cycle of an Arabidopsis cell comprising the steps of:
a.) producing at a particular time point after the release from the cell cycle block a sample of a synchronised Arabidopsis cell suspension culture obtainable through the method of claim 41, wherein said cell suspension culture has a maximum labelling index of at least 15% or a maximum mitotic index of at least 9% when cells of said suspension culture are progressing through the cell cycle; and
b.) analyzing the relative level of a particular metabolite in cells of the sample.
No fee is deemed necessary in connection with the filing of this Preliminary Amendment. If any fee is required, authorization is hereby given to charge any such fee to Deposit Account No. 01-1785.
